(19) United States
(12) Patent Application Publication
(10) Pub. No.: US 2025/0288507 A1
(43) Pub. Date: Sep. 18, 2025

(54) ZINC-BASED MATERIALS FOR TREATMENT OF DENTAL CARIES

(71) Applicant: New York University, New York, NY (US)

(72) Inventor: Marc A. WALTERS, New Rochelle, NY (US)

(21) Appl. No.: 18/860,946

(22) PCT Filed: Apr. 27, 2023
(86) PCT No.: PCT/US2023/066328

§ 371 Date: Oct. 28, 2024

Related U.S. Application Data
(60) Provisional application No. 63/335,634, filed on Apr. 27, 2022.
(60) Provisional application No. 63/479,982, filed on Jan. 13, 2023.
(60) Provisional application No. 63/483,493, filed on Feb. 6, 2023.

(51) Int. Cl. A61K 8/27; A61K 8/21; A61K 8/24; A61Q 11/00

(52) U.S. Cl. CPC A61K 8/27; A61K 8/21; A61K 8/24; A61Q 11/00; A61K 2800/58; A61K 2800/92

(57) ABSTRACT

Provided is compositions and methods for treating dental caries in individuals and animals. The compositions may be zinc compositions of [Zn(NH₃)₄]F₂. Also provided are methods of making inorganic zinc compounds (e.g., ZnO, Zn(OH)₂, or Zn(OH)F) onto the surface of a carious lesion. The deposition of inorganic zinc compound (e.g., ZnO, Zn(OH)₂, or Zn(OH)F) may occur from dilution of a Zn²⁺/NH₃ solution (e.g., an aqueous or alcohol-based solution) into water (e.g., deionized water). The formation of inorganic zinc compound (e.g., ZnO, Zn(OH)₂, or Zn(OH)F) may be instantaneous upon dilution.

### CROSS REFERENCE TO RELATED APPLICATIONS

This application claims priority to U.S. Provisional Applications 63/335,634, 63/479,982, and 63/483,493, filed on Apr. 27, 2022, Jan. 13, 2023, and Feb. 6, 2023, respectively, the disclosures of which are incorporated by reference herein.

### BACKGROUND OF THE DISCLOSURE

Traditionally dental caries are treated by the surgical removal of infected tissue where the biological mineral, hydroxyapatite (HAp), Ca₁₀(PO₄)₆(OH)₂, has been eroded by acid produced by cariogenic bacteria, mainly acidogenic and aciduric, in the oral cavity. The resulting preparations are then restored with a variety of materials. For early carious lesions, a surgical approach has increasingly been replaced by minimally invasive dentistry (MID) protocols to arrest the lesion progression and promote remineralization to repair dentin that is damaged, but structurally intact.

Silver diamine fluoride (SDF) has been used in MID for the treatment of early dental caries. However a serious drawback of the SDF modality is that it produces a profound dark stain, which is problematic for aesthetics and patient acceptance. It is generally recognized that SDF reacts with exposed dentin to form a silver phosphate, Ag₃(PO₄), a semiconductor that undergoes autoreduction under visible light exposure. The result is the formation of neutral silver atoms that assemble into silver nanoparticles. These nanoparticles intrinsically absorb in the violet (yellow colored colloidal phases) but then form aggregates that absorb broadly across the visible spectrum and therefore appear black. While the time course for the formation of black deposits is variable, it is certain to occur whenever SDF is employed at its clinically recommended concentration of 38% (w/w) in water. The aims of MID would be well served by a topical agent that is generally acceptable to parents of pediatric patients, who as a class are perhaps the major beneficiaries of SDF treatment. Unfortunately, parents are reluctant to accept the treatment because of the stain formation, particularly on anterior teeth.

To solve the problem of staining a number of chemical agents have been tested, such as ammonium hexafluorosilicate, zinc fluoride, and potassium iodide. The application of SDF, immediately followed by saturated aqueous KI has been recommended for clinical use. However, it has been reported that SDF/KI precipitates significantly reduced the bond strengths of glass ionomer and composite restorations. Moreover, even after treatment with SDF/KI teeth have been reported to change color within a period of 14 days. The SDF/KI treatment option costs fifteen times more than the SDF unit dose treatment option. Most importantly, KI is contraindicated for use during pregnancy as it is associated with congenital iodide goiter. The many caveats associated with SDF point toward the need for an alternative.

ZnO has been shown to be effective against Strep. mutans. Studies have shown that ZnO transforms in saliva to hopeite, which then serves as a reservoir of Zn²⁺ to inhibit bacterial proliferation and caries progression.

Water dispersible ZnO has been described. The delivery of antibacterial agents in high concentration favors the formation of a chemical barrier to bacterial incursion and proliferation. However, the retention of water dispersions of ZnO in carious tissue is challenging as there is no obvious route for the transformation of a ZnO dispersion to a mechanically stable mineral phase.

There are several routes to the desired minerals. The preparation of 1.25 M ethanol rich solutions of Zn²⁺ in the form of ZnO nanoparticles has been reported. A ZnO nanoparticle dispersion has been reported by using triethanolamine as a capping agent. It was also shown that nanoparticles readily aggregate in electrolyte containing solutions, which could be crucial for the deposition of ZnO in dentin tubules. Once deposited, the low solubility nanoparticles would be expected to undergo a slow dissolution accompanied by the transformation of ZnO to hopeite Zn₃(PO₄)₂ and the uptake of Zn²⁺ in the biological HAP matrix of the tubules. The incorporation of zinc has been shown to benefit the stabilization of both dentin and enamel against bacterial incursion.

### SUMMARY OF THE DISCLOSURE

In an aspect, the present disclosure provides a method to treat dental caries comprising application of compositions that result in the deposition of Zn₃(PO)₄ and/or (NH₄)ZnPO₄ and/or application of a zinc species comprising fluoride (e.g., [Zn(NH₃)₄]F₂) that results in the deposition of an inorganic zinc compound.

In an aspect, the present disclosure provides methods of deposition an inorganic zinc compound. In various embodiments, the inorganic zinc compound is ZnO, Zn(OH)₂, or Zn(OH)F. The inorganic zinc compound (e.g., ZnO, Zn(OH)₂, or Zn(OH)F) may be deposited onto the surface of a carious lesion. The deposition of inorganic zinc compound (e.g., ZnO, Zn(OH)₂, or Zn(OH)F) may occur from dilution of a Zn²⁺/NH₃ solution (e.g., an aqueous or alcohol-based solution) into water (e.g., deionized water). The formation of inorganic zinc compound (e.g., ZnO, Zn(OH)₂, or Zn(OH)F) may be instantaneous upon dilution.

### BRIEF DESCRIPTION OF THE FIGURES

For a fuller understanding of the nature and objects of the disclosure, reference should be made to the following detailed description taken in conjunction with the accompanying figures.

FIG. 1 shows BSE SEM micrographs of the milled HAp surface samples. (A) Zinc nitrate alone, (B) zinc nitrate followed by dibasic ammonium phosphate, (C) ammonium phosphate followed by zinc nitrate, (D) control.

FIG. 2 shows SEM micrographs of the milled HAp surface samples with EDS data showing calcium (red) and zinc (blue). (A) zinc nitrate alone, (B) zinc nitrate followed by dibasic ammonium phosphate, (C) ammonium phosphate followed by zinc nitrate, (D) control.

FIG. 3 shows BSE SEM micrographs of tooth #2 dentin cross-section with and without EDS data showing calcium (blue) and zinc (yellow). (A and B) Wide field view 204.8 μm. The green box shows the area selected for the narrow field image. (C and D) Narrow field at 62.5 μm.

FIG. 4 shows BSE SEM micrographs of tooth #3 dentin cross-section with and without EDS data showing calcium (blue) and zinc (yellow). (A and B) Wide field view 204.8 μm. The green box shows the area selected for the narrow field image. (C and D) Narrow field at 62.5 μm.

FIG. 5 shows sample substrate consists of milled HAp bonded by epoxy to a support of silicon protected by a layer of Scotch tape.

FIG. 6 shows zinc ammonium phosphate powder, Zn(NH₄)PO₄ (A), milled hydroxyapatite (HAp) powder, calcium hydroxyapatite on epoxy, (B) HAp powder treated with zinc nitrate followed by ammonium phosphate, dibasic (C), HAp powder on epoxy treated with ammonium phosphate dibasic followed by zinc nitrate (D) hopeite, Zn₃(PO₄)₂, technical grade (E).

FIG. 7 shows (A) mineral formation from dibasic ammonium phosphate solution added to a thin layer of zinc solution on HAp, scale bar 5.0 μm, (B) magnified, scale bar 1.0 μm. (C) Mineral formation from zinc nitrate solution added to a thin layer of dibasic ammonium phosphate solution on HAp, scale bar 5.0 μm (D) magnified, scale bar 1.0 μm.

FIG. 8 shows zinc ammonium phosphate powder after an eleven-day exposure to (top) artificial saliva, (middle) DI water, (bottom) hopeite calculated pattern 34869-ICSD.

FIG. 9 shows hopeite powder diffraction pattern (bottom) hopeite standard, calculated 34869-ICSD, ( . . . ) hopeite soaked in water one week, (-) hopeite soaked in artificial saliva one week.

FIG. 10 shows early studies on zinc iodide established methods that we have employed or modified for the preparation of solutions containing zinc and fluoride. Dissolution in aqueous ammonia is well known and convenient. Reaction of ZnI₂ and ZnBr₂ with gas phase ammonia produces crystals that have been characterized by X-ray crystallography. More recently, a series of zinc amines produced by the reaction of ZnF₂ in supercritical ammonia were shown.

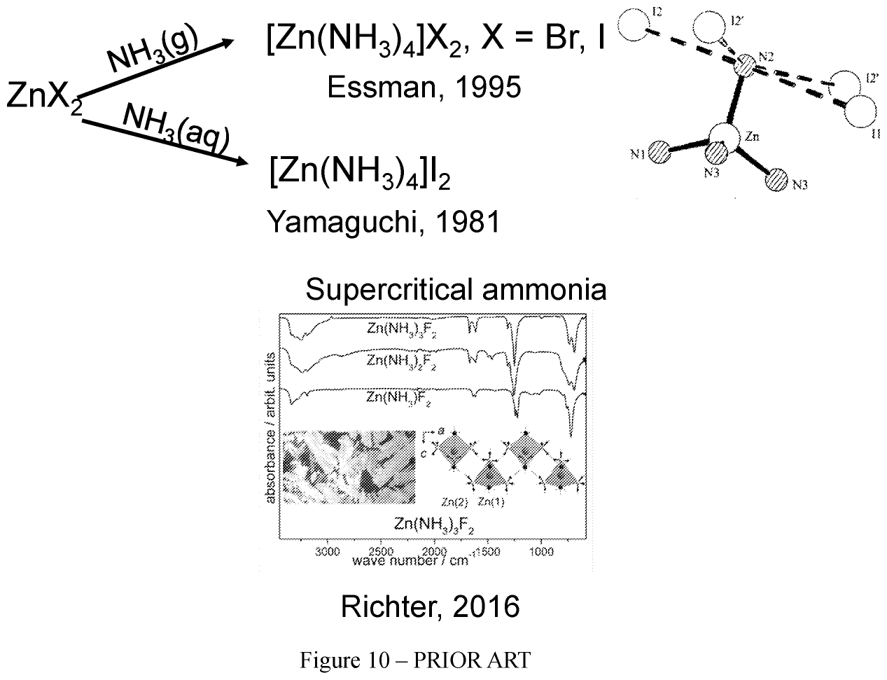

FIG. 11 shows the formation of various zinc species from [Zn(NH₃)₄]F₂.

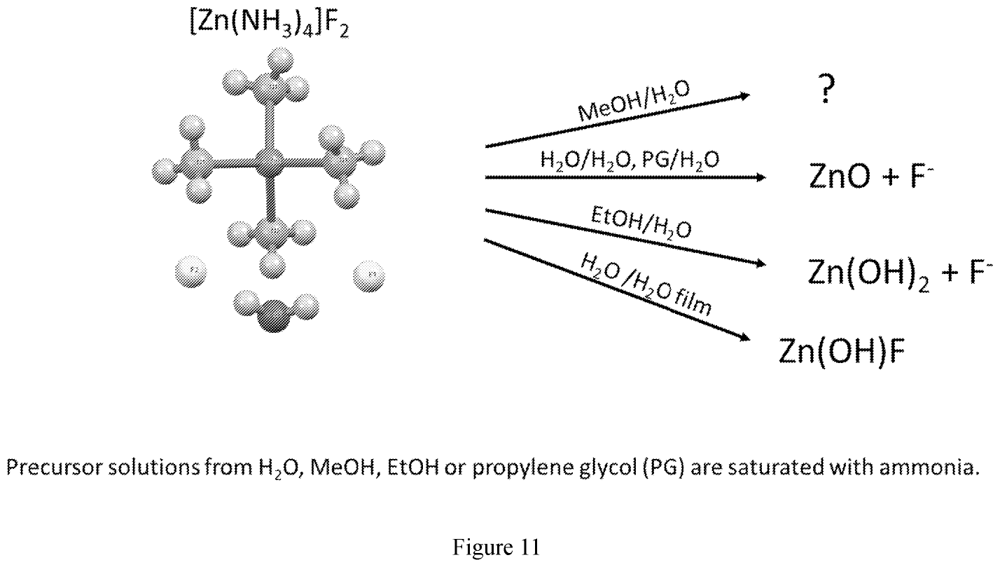

FIG. 12 shows powder diffraction pattern of the product ZnO from the addition of [Zn(NH₃)₄]F₂(alc) to water. alc=propylene glycol.

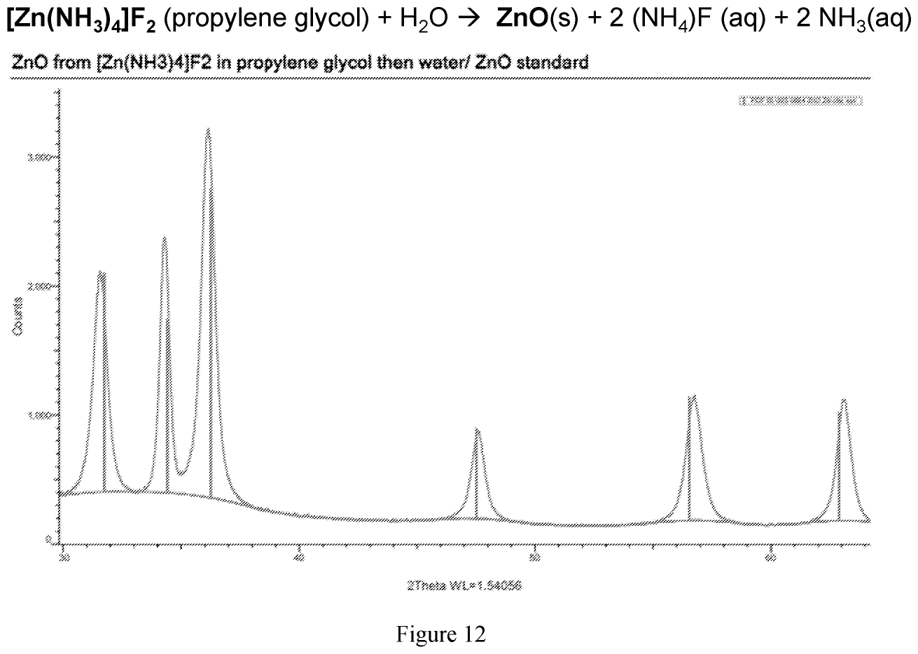

FIG. 13 shows SEM micrograph of ZnO from the addition of [Zn(NH₃)₄]F₂(alc) to water. alc=propylene glycol.

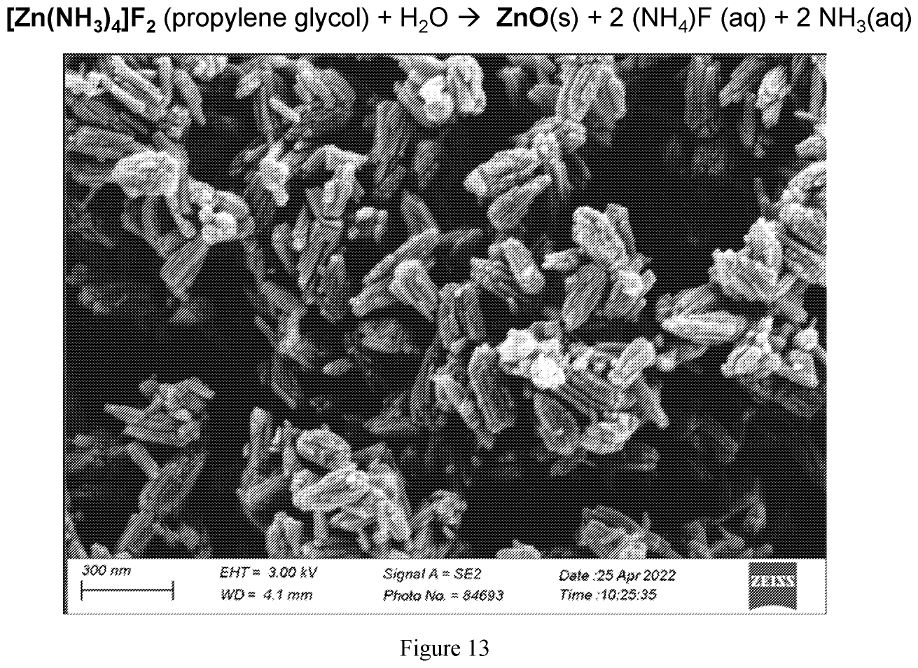

FIG. 14 shows mineral compounds relevant to Streptococcus mutans. Low solubility and conversion of incipient minerals to increasingly stable mineral sources of zinc is the goal.

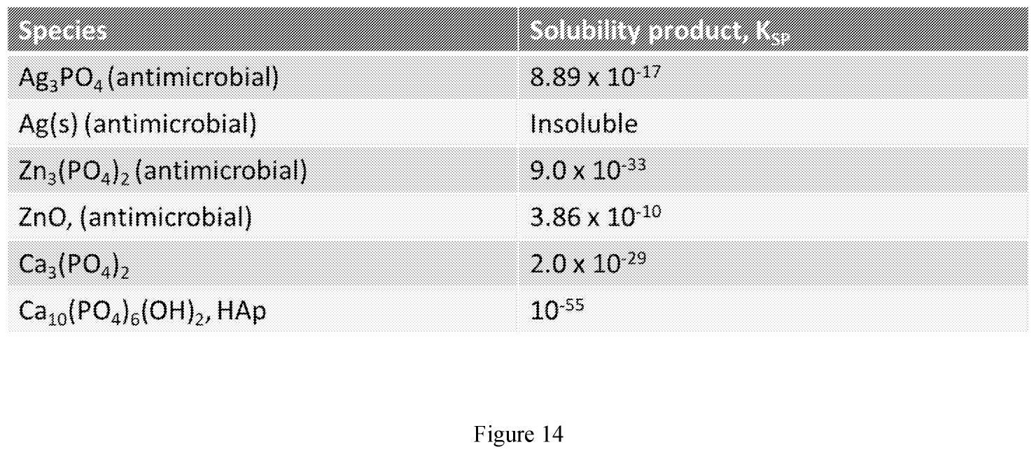

FIG. 15 shows hopeite, which is intrinsically colorless and potentially antimicrobial after deposition in dentin.

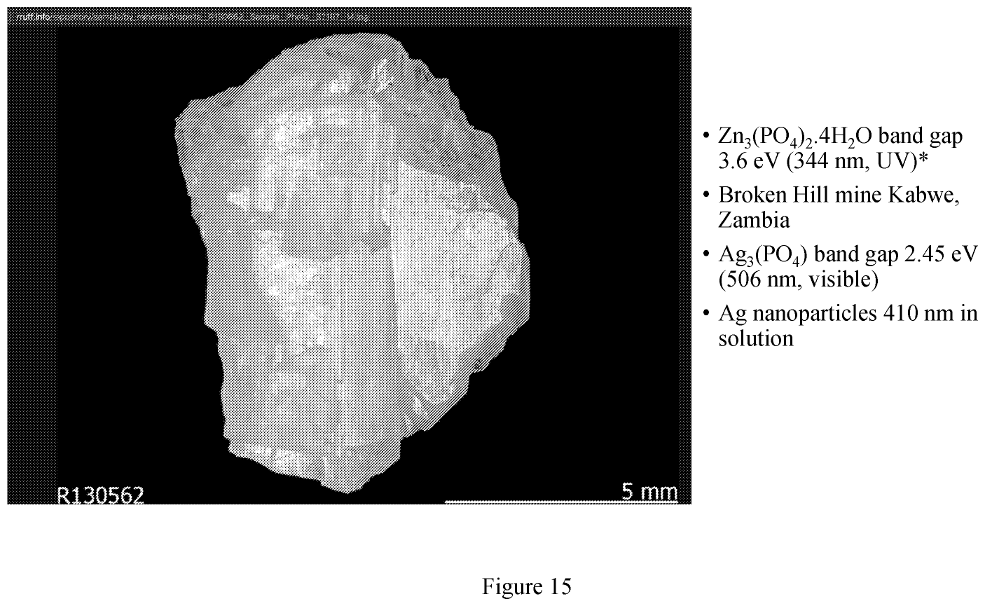

FIG. 16 shows preparation for scanning electron microscopic imaging the synthetic hydroxyapatite (HAp), model of dentin tubule surface.

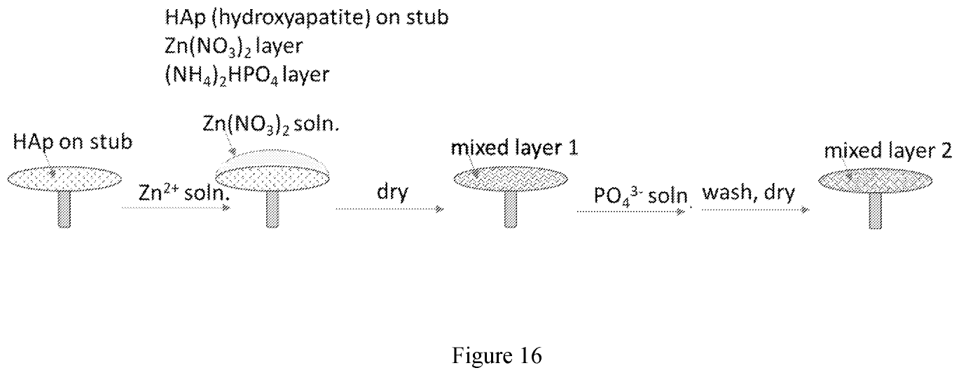

FIG. 17 shows (left) x-ray diffraction and (right) a scanning electron micrograph of a deposit from a zinc solution, followed by application of (NH₄)₂HPO₄ (aq).

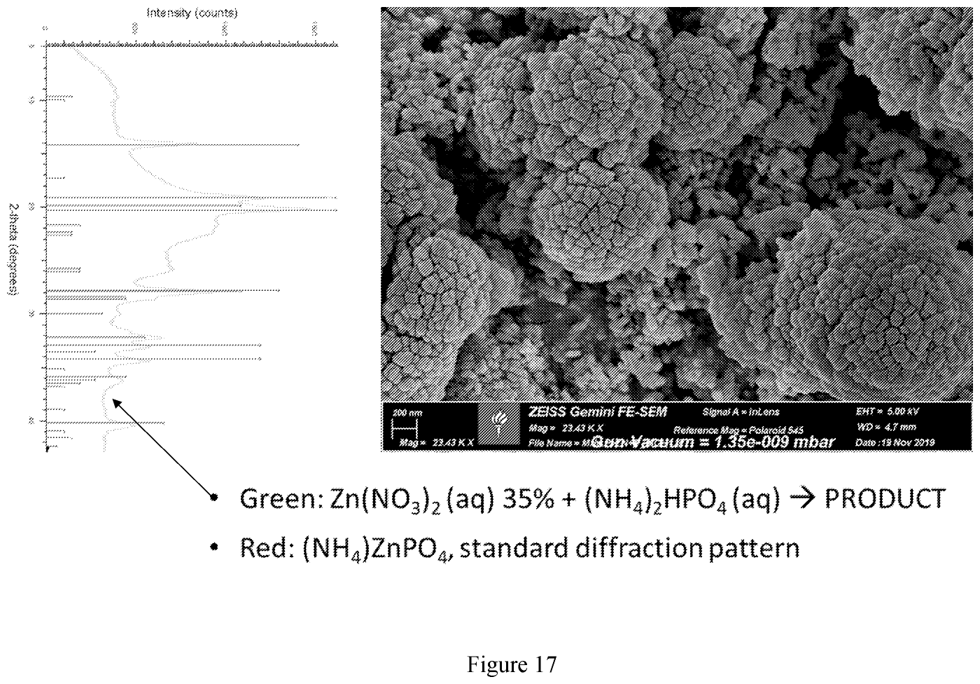

FIG. 18 shows (left) x-ray diffraction and (right) a scanning electron micrograph of a deposit from zinc phosphate soaked in water.

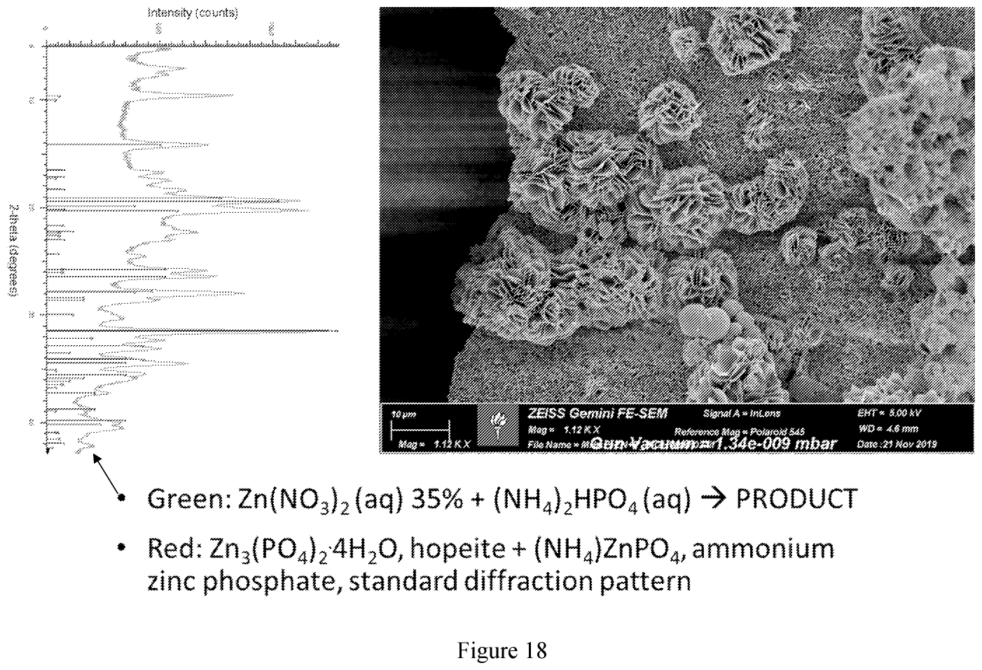

FIG. 19 shows the ellipsoid plot for F₂H₁₈N₄O₃Zn, which is [Zn(NH₃)₄]F₂.

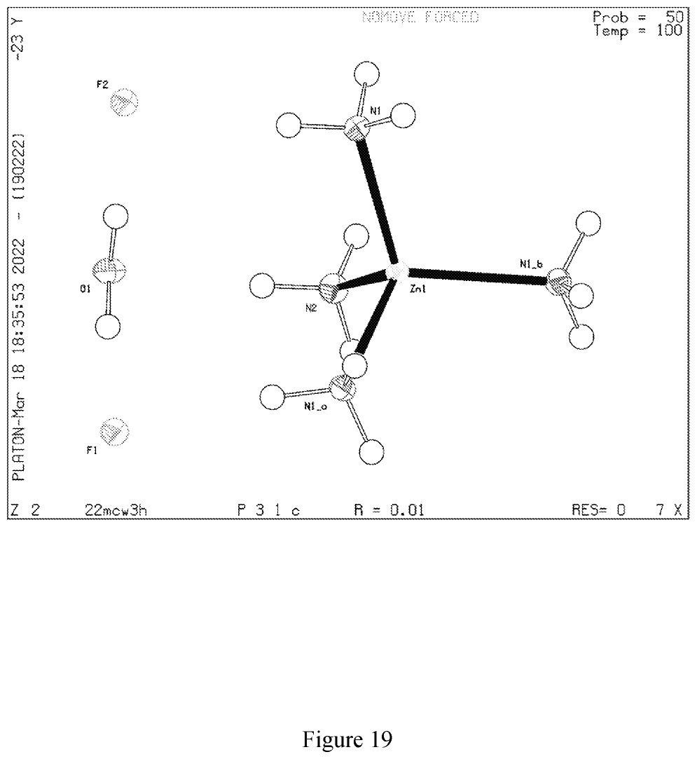

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The present disclosure provides a safe, non-staining, anti-caries agent as an alternative to SDF. Embodiments for this agent include antibacterial activity, the potential to occlude dentin tubules, which may alleviate dentin hypersensitivity, and the promotion of tissue remineralization. A zinc-based mineral is well suited for this purpose. Proposed mechanisms for antibacterial action of zinc ions include: 1) binding to microorganism membranes causing bacterial membrane rupture and leakage, 2) prolonging the growth cycle and slowing cellular division of bacteria, and 3) an affinity for gram-positive bacteria, which includes Streptococcus mutans, a primary bacterium in the caries forming process.

The potential antimicrobial benefits of zinc deposition have been detailed in several recent studies. One study demonstrated the use of zinc phosphate, as a titanium implant coating that was shown to be biocompatible, antibacterial, and a promoter of hydroxyapatite (Hap) formation. Another recent study showed that Zn²⁺ complexes and ZnO nanoparticles are active against Streptococcus mutans. Additionally, it has been shown that zinc loaded HAp nanorods are antibacterial against several organisms including Streptococcus mutans.

One focus is on the direct delivery of zinc phosphate to dentin tubules. Zinc phosphate has had a long history in dentistry and is considered to be a safe and reliable luting agent that only recently has been replaced by modern agents with significantly increased mechanical properties and adhesive strength. Yet, zinc phosphate minerals may prove efficacious for the treatment of caries. Investigators have demonstrated that zinc phosphate coatings can improve hemocompatibility, cytocompatibility, and antibacterial activity compared to pure biodegradable zinc which is thought to release zinc ions too rapidly.

The challenge is to deploy zinc phosphate in dentin as an antibacterial barrier. In the following we describe the deposition of zinc phosphate mineral that effectively occludes dentin tubules with crystallization at the tubule openings and by the assembly of crystals within the tubules.

In an aspect, the present disclosure provides a method to treat dental caries comprising application of compositions that result in the deposition of Zn₃(PO)₄ and/or (NH₄)ZnPO₄ and/or application of a zinc species comprising fluoride (e.g., [Zn(NH₃)₄]F₂) that results in the deposition of an inorganic zinc compound.

Zn₃(PO)₄ and/or (NH₄)ZnPO₄ may be formed by the mixing of various zinc salts and phosphate salts. For example, dental caries may be treated by applying a composition comprising a zinc salt, followed by application of composition comprising a phosphate salt. In various other embodiments, one or more dental caries may be treated by applying a composition comprising a phosphate salt, followed by application of composition comprising a zinc salt.

Various zinc salts may be used. For example, the zinc salt is Zn(NO₃)₂, ZnSO₄, Zn(OAc)₂, ZnF₂, ZnCl₂, ZnBr₂, ZnI₂, Zn(NH₃)₄]F₂, or the like, or various combinations thereof.

Various phosphate salts may be used. For example, the phosphate salt is (NH₄)₂HPO₄, (NH₄)H₂PO₄, K₃PO₄, K₂HPO₄, KH₂PO₄, (NH₄)₂PO₃F, or the like, or various combinations thereof.

Without intending to be bound by any particular theory, it is considered when a composition comprising Zn(NO₃)₂ is applied followed by the application of a composition comprising (NH₄)₂HPO₄, (NH₄)₂ZnPO₄ is the species predominantly formed. Without intending to be bound by any particular theory, it is considered when a composition comprising (NH₄)₂HPO₄ is applied followed by the application of a composition comprising Zn(NO₃)₂, Zn₃(PO₄)₂ or Zn₃(PO₄)₂·4H₂O (i.e., hopeite) is the species predominantly formed. Without intending to be bound by any particular theory, it is considered that (NH₄)ZnPO₄ will convert to hopeite over time.

In various embodiments, the composition comprises [Zn(NH₃)₄]F₂. A composition comprising [Zn(NH₃)₄]F₂ may further comprise a liquid medium. The liquid medium may be an alcohol (such as, for example, a glycol) and/or water. For example, if the alcohol is a glycol, the glycol can be propylene glycol. In various embodiments, when the glycol is propylene glycol, the liquid medium may further comprise water. For example, the alcohol can be ethanol or methanol. In various embodiments, when the alcohol is ethanol, the liquid medium may further comprise water. In various embodiments, when the alcohol is methanol, the liquid medium may further comprise water. The medium may further comprise ammonia. If the medium is methanol, the ammonia concentration may be 7 N. If the medium is ethanol, the ammonia concentration may be 2 N. If the medium is water, the ammonia concentration may be 30% ammonia (by weight). If the medium is propylene glycol, the propylene glycol may be saturated with ammonia at STP.

The composition may be applied to a surface of a tooth having one or more dental caries and the [Zn(NH₃)₄]F₂ is formed prior to the application to the surface of the tooth or directly to the dental caries. Without intending to be bound by any particular theory, after application of [Zn(NH₃)₄]F₂ it is considered that an inorganic zinc compound (e.g., ZnO, Zn(OH)₂, or Zn(OH)F) will be formed over time and allow for the gradual release of fluoride ions.

The resulting Zn-based materials (e.g., (NH₄)ZnPO₄, Zn₃(PO₄)₂, Zn₃(PO₄)₂·4H₂O, [Zn(NH₃)₄]F₂) and are insoluble and have desirable crystalline properties and antibacterial properties as described in the Examples presented herein.

In an aspect, the present disclosure provides methods of deposition an inorganic zinc compound. In various embodiments, the inorganic zinc compound is ZnO, Zn(OH)₂, or Zn(OH)F. The inorganic zinc compound (e.g., ZnO, Zn(OH)₂, or Zn(OH)F) may be deposited onto the surface of a carious lesion. The deposition of inorganic zinc compound (e.g., ZnO, Zn(OH)₂, or Zn(OH)F) may occur from dilution of a Zn²⁺/NH₃ solution (e.g., an aqueous or alcohol-based solution) into water (e.g., deionized water). The formation of inorganic zinc compound (e.g., ZnO, Zn(OH)₂, or Zn(OH)F) may be instantaneous upon dilution.

In various embodiments, the method comprises inorganic zinc compound (e.g., ZnO, Zn(OH)₂, or Zn(OH)F) deposition from solutions of Zn²⁺ salts in aqueous ammonia. Non-limiting examples of Zn²⁺ salts include ZnF₂, Zn(NO₃)₂, ZnSO₄, ZnCl₂, ZnBr₂, ZnI₂, and combinations thereof. The concentration of Zn²⁺ prior to dilution may be 1 to 5 M, including every 0.1 value and range therebetween (e.g., 3.0 M). The concentration of ammonia (NH₃) may be 10-30% by weight (relative to the total weight of water and ammonia), including all ranges and values therebetween (e.g., 10%, 20%, or 30% by weight). The molar ratio of NH₃:Zn²⁺ in the solution may be 1:1-10:1 (e.g., 1:1, 2:1, 3:1, 4:1, 5:1, 6:1, 7:1, 8:1, 9:1, or 10:1). In various examples, the molar ratio of NH₃:Zn²⁺ 5.5:1.

The formation of the inorganic zinc compound may be altered by the choice of solution comprising Zn²⁺/NH₃. For example, if the Zn²⁺/NH₃ is in water prior or propylene glycol prior to dilution, ZnO will form upon dilution. In another example, if the Zn²⁺/NH₃ is in ethanol and water prior to dilution, Zn(OH)₂ will form upon dilution. In another example, if the Zn²⁺/NH₃ is in a water film prior to dilution, Zn(OH)F will form upon dilution.

The formation of an inorganic zinc compound occurs after dilution of Zn²⁺/NH₃(aq) into water (e.g., deionized water). For example, the dilution should be approximately 20× or greater (i.e., 50 μL added to 1 mL of water (e.g., deionized water)). The dilution into water may occur directly on the surface of the tooth. For example, concentrated Zn²⁺/NH₃(aq) is diluted and the concentration of diluted Zn²⁺/NH₃(aq) is at least 20 fold with water.

In various embodiments, the method comprises inorganic zinc compound deposition (e.g., ZnO, Zn(OH)₂, or Zn(OH)F) from solutions of Zn²⁺ salts in ammonia dissolved in an alcohol solvent. Non-limiting examples of alcohols include methanol, ethanol, N-propanol, isopropanol, butanol, sec-butanol, tert-butanol, propylene glycol, ethylene glycol, and combinations thereof. Examples of Zn²⁺ include but are not limited to ZnF₂, Zn(NO₃)₂, ZnSO₄, ZnCl₂, ZnBr₂, ZnI₂, and combinations thereof. The concentration of Zn²⁺ prior to dilution may be 1 to 5 M, including every 0.1 value and range therebetween (e.g., 3.0 M). The concentration of ammonia may be 1-7 N, including all values and ranges therebetween (e.g., 1, 2, 3, 4, 5, 6, or 7 N). The molar ratio NH₃:Zn²⁺ in the solution may be 1:1-10:1, including every value and range therebetween (e.g., 1:1, 2:1, 3:1, 4:1, 5:1, 6:1, 7:1, 8:1, 9:1, or 10:1). In various examples, the molar ratio of NH₃:Zn²⁺ 5.5:1.

The formation of an inorganic zinc compound (e.g., ZnO, Zn(OH)₂, or Zn(OH)F) occurs after dilution of Zn²⁺/NH₃ (alcohol) into water (e.g., deionized water). For example, the dilution should be approximately 20× (i.e., 50 μL added to 1 mL of water (e.g., deionized water)).

The formation of ZnO, Zn(OH)₂, or Zn(OH)F at a carious lesion where Zn²⁺/NH₃(aq) or Zn²⁺/NH₃ (alc) may be deposited as an antimicrobial on a carious lesion and/or as a tubule occluding agent on a carious lesion.

The compositions of the present disclosure may be applied to the surface of a tooth of an individual by various means. In various embodiments, the compositions are brushed onto/into the one or more dental caries.

Any method of the present disclosure may be performed on an individual in need of treatment, where the individual has one or more dental caries. The individual in need of treatment may be a human or non-human mammal. Non-limiting examples of non-human mammals include cows, pigs, rats, cats, dogs, other agricultural animal, pet, service animals, and the like.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various embodiments of the present disclosure.

Statement 1. A method for treating one or more dental caries comprising: i) applying to the one or more dental caries a composition comprising a zinc salt and applying to the one or more dental caries a composition comprising a phosphate salt, wherein Zn₃(PO)₄ (and/or its hydrate) and/or (NH₄)ZnPO₄ are formed or ii) applying to the one or more dental caries a composition comprising zinc and fluoride (e.g., wherein an inorganic zinc compound is formed).
Statement 2. A method according to Statement 1, wherein the applying is by brushing the composition onto a surface of the tooth having the dental caries.
Statement 3. A method according to Statement 1 or Statement 2, wherein the zinc salt is Zn(NO₃)₂, ZnSO₄, Zn(OAc)₂, ZnF₂, ZnCl₂, ZnBr₂, ZnI₂, Zn(NH₃)₄]F₂, or the like, or various combinations thereof.
Statement 4. A method according to any one of the preceding Statements, wherein the phosphate salt is (NH₄)₂HPO₄, (NH₄)H₂PO₄, K₃PO₄, K₂HPO₄, KH₂PO₄, (NH₄)₂PO₃F, or the like, or various combinations thereof.
Statement 5. A method according to any one of the preceding Statements, wherein the composition comprising the zinc salt is applied prior to the composition comprising the phosphate salt.
Statement 6. A method according to any one of the preceding Statements, wherein the composition comprising the phosphate salt is applied prior to the composition comprising the zinc salt.
Statement 7. A method according to any one of the preceding Statements, wherein the concentration of the zinc salt is 30-50 wt % (e.g., 30, 31, 32, 33, 34, 35, 36, 37, 38, 39, 40, 41, 42, 43, 44, 45, 46, 47, 48, 49, or 50 wt %).
Statement 8. A method according to Statement 7, wherein the concentration of the zinc salt is 35-45 wt %, relative to the total weight of the composition.
Statement 9. A method according to Statement 8, wherein the concentration of the zinc salt is 38-43 wt %, relative to the total weight of the composition.
Statement 10. A method according to any one of the preceding Statements, wherein the concentration of the phosphate salt is 10-40 wt % (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 wt %), relative to the total weight of the composition.
Statement 11. A method according to Statement 10, wherein the concentration of the phosphate salt is 15-35 wt %, relative to the total weight of the composition.
Statement 12. A method according to Statement 11, wherein the concentration of the phosphate salt is 20-30 wt %, relative to the total weight of the composition.
Statement 13. A method according to any one of the preceding Statements, wherein Zn₃(PO)₄ (and/or its hydrate) and/or (NH₄)ZnPO₄ are formed.
Statement 14. A method according to any one of the preceding Statements, wherein the composition comprising zinc and fluoride comprises [Zn(NH₃)₄]F₂.
Statement 15. A method according to Statement 14, wherein the composition further comprises a liquid medium.
Statement 16. A method according to Statement 15, wherein the liquid medium comprises an alcohol, a glycol, and/or water.
17. A method according to Statement 16, wherein the glycol is propylene glycol.
Statement 18. A method according to Statement 17, wherein the composition further comprises water.
Statement 19. A method according to Statement 16, wherein the alcohol is methanol or ethanol.
Statement 20. A method according to Statement 19, wherein the alcohol is methanol.
Statement 21. A method according to Statement 20, wherein the composition further comprises water.
Statement 22. A method according to Statement 19, wherein the alcohol is ethanol.
Statement 23. A method according to Statement 22, wherein the composition further comprises water.
Statement 24. A composition comprising [Zn(NH₃)₄]F₂.
Statement 25. A composition according to Statement 24, wherein the composition further comprises a liquid medium.
Statement 26. A composition according to Statement 25, wherein the liquid medium comprises an alcohol, a glycol, and/or water.
Statement 27. A composition according to Statement 26, wherein the glycol is propylene glycol.
Statement 28. A composition according to Statement 27, wherein the composition further comprises water.
Statement 29. A composition according to Statement 26, wherein the alcohol is methanol or ethanol.
Statement 30. A composition according to Statement 29, wherein the alcohol is methanol.
Statement 31. A composition according to Statement 30, wherein the composition further comprises water.
Statement 32. A composition according to Statement 29, wherein the alcohol is ethanol.
Statement 33. A composition according to Statement 32, wherein the composition further comprises water.
Statement 34. A method for preparing an inorganic zinc compound comprising: diluting with water a solution comprising Zn²⁺/NH₃, wherein the dilution results in the formation of ZnO, Zn(OH)₂, and/or Zn(OH)F.

Statement 35. A method according to Statement 34, wherein the solution comprising Zn²⁺/NH₃ is an aqueous ammonia solution.

Statement 36. A method according to Statement 34 or Statement 35, wherein the Zn² is from ZnF₂, Zn(NO₃)₂, ZnSO₄, ZnCl₂, ZnBr₂, ZnI₂, or a combination thereof.
Statement 37. A method according to any one of Statements 34-36, wherein the concentration of Zn²⁺ is 1 to 5 M (e.g., 3 M).
Statement 38. A method according to any one of Statements 34-37, wherein the concentration of ammonia is 10 to 30% by weight relative to the total weight of water and ammonia.
Statement 39. A method according to any one of Statements 34-38, wherein the molar ratio of NH₃:Zn²⁺ in the solution is 1:1-10:1.
Statement 40. A method according to Statement 34, wherein the solution comprising Zn²⁺/NH₃ is an alcohol-based ammonia solution.
Statement 41. A method according to Statement 40, wherein the alcohol of the alcohol-based ammonia solution is chosen from methanol, ethanol, n-propanol, isopropanol, butanol, sec-butanol, tert-butanol, propylene glycol, ethylene glycol, and combinations thereof.
Statement 42. A method according to Statement 40 or Statement 41, wherein the Zn²⁺ is from ZnF₂, Zn(NO₃)₂, ZnSO₄, ZnCl₂, ZnBr₂, ZnI₂, or a combination thereof.
Statement 43. A method according to any one of Statements 34 or 40-42, wherein the concentration of Zn²⁺ is 1 to 5 M (e.g., 3 M).
Statement 44. A method according to any one of Statements 40-43, wherein the concentration of ammonia is 1 to 7 N.
Statement 45. A method according to any one of Statements 40-44, wherein the molar ratio of NH₃:Zn²⁺ in the solution is 1:1-10:1.
Statement 46. A method according to any one of Statements 40-45, wherein the solution comprising Zn²⁺/NH₃ is applied onto a surface of a tooth or caries of the tooth prior to dilution.
Statement 47. A method according to any one of Statements 40-46, wherein the solution comprising Zn²⁺/NH₃ is diluted at least 20× with water on the surface of the tooth or the caries of the tooth.
Statement 48. A method according to any one of Statements 40-47, wherein the method occurs in the mouth of an individual.

The following examples are presented to illustrate the present disclosure. They are not intended to be limiting in any matter.

### Example 1

This example provides a description of methods of making and using a zinc-based material of the present disclosure.

Dentin blocks prepared from human permanent molars were treated topically by the sequential application of aqueous zinc nitrate and aqueous ammonium phosphate salt solutions. Field emission-scanning electron microscopy (FE-SEM) images and energy dispersive X-ray scattering (EDS) showed extensive crystalline and colorless zinc mineral deposits both within dentin tubules and on the dentin surface. Parallel experiments on the surface of milled hydroxyapatite (HAp) with XRD analysis suggest that, depending on reaction conditions, the initial mineral deposited can be either zinc ammonium phosphate (NH₄)ZnPO₄ or zinc phosphate, Zn₃(PO₄)₂. The results provide a path to optimize the direct deposition of ionic zinc in dentin tubules in carious lesions where it can serve as an antimicrobial agent for caries arrest, possible remineralization of lesions and mitigation of hypersensitivity.

Two experimental designs were utilized herein. In the first, solutions were applied to non-carious dentin and both the surface and tubule cross-sections were evaluated with SEM and EDS analysis. In the second, solutions were applied to milled HAp coated layers on silicon substrates and data was collected utilizing scanning electron microscopy (SEM), energy-dispersive X-ray spectroscopy (EDS), and X-ray powder diffraction (XRD).

Milled HAp was donated. The zinc nitrate, Zn(NO₃)₂·6 H₂O, was purchased from Strem chemicals. Zinc phosphate, Zn₃(PO₄)₂·xH₂O, was purchased from Alfa Aesar (Lot: M09E004, x=3.64). Monobasic ammonium phosphate (NH₄)H₂PO₄, was purchased from Honeywell/Fluka (Lot: J0930) and dibasic ammonium phosphate (NH₄)₂HPO₄, was purchased from Fischer Chemicals (Lot: 972377). Ammonium hydroxide, ACS (28.0-30.0% NH₃) was purchased from Alfa Aesar (Lot: P05F004). Phosphate buffer saline (PBS) was purchased from Fisher Scientific, Waltham, MA, USA. All chemicals were used as received.

Stock solutions of 42% (w/w) zinc nitrate hexahydrate and 24% (w/w) dibasic ammonium phosphate were prepared in deionized (DI) water, in 5 ml for each solution, for use in experiments described below. Note that the mole ratio of the reactants would be 1:1 if the solutions were directly combined in equal mass. The epoxy used to affix HAp powder to the substrate was Devcon 5 Minute® Epoxy, Clear. Artificial saliva (1700-0305) was purchased from Pickering Laboratories and used as received. Artificial saliva 1700-0305: (Pickering Laboratories); Sodium Carboxymethyl Cellulose 10.00 g/L; Potassium Phosphate Monobasic 0.326 g/L; Potassium Chloride 0.625 g/L; Potassium Phosphate Dibasic 0.804 g/L, Magnesium Chloride hexahydrate 0.059 g/L; Calcium Chloride dehydrate 0.166 g/L; Methyl-p-hydroxybenzoate 2.00 g/L.

Synthesis of zinc ammonium phosphate. Zinc ammonium phosphate, (NH₄)ZnPO₄, was synthesized by the method of Baitahe with a modification. Briefly 5.97 g of Zn(NO₃)₂·6H₂O was weighed into a 40 ml beaker. To this 21 ml of 1M (NH₄)H₂PO₄ was added with stirring followed by 20 ml of 2M NH₃ (aq, 28.0-30.0%). The resulting slurry was stirred vigorously for 15 min. The product was then collected by filtration, washed with DI water and transferred to a watch glass to dry. As a modification, an aging step was added in which the product was resuspended and stirred overnight in DI water, collected by filtration, washed with acetone, followed by ethyl ether and allowed to dry. The resulting powder was moderately free flowing. XRD analysis showed the product scan to match that of previously reported zinc ammonium phosphate, 85444-ICSD.

Dentin samples: Preparation and application of experimental solutions. Ethical approval for the use of extracted human teeth was obtained in accordance with guidelines of the New York University College of Dentistry. Four extracted sound human third molars were stored at 4° C. in a 0.1% thymol solution to prevent microbial growth after extraction. The teeth were then removed from the thymol solution, immersed for 30 min in DI water, and then rinsed with DI water. The teeth were then immersed in 1×PBS that was diluted from a 10×PBS solution using DI water. Teeth were left in the 1×PBS solution for 7 days prior to experimentation.

All teeth were sectioned with a Buehler Isomet diamond blade (Lake Bluff, IL, USA) tangentially across the occlusal surface approximately 1 mm deep to the shortest cusp tip to create a flat occlusal surface. The cusp tips were discarded. The teeth were then sectioned approximately 2 mm apical to the deepest depth of the occlusal central groove to remove occlusal enamel and expose the underlying dentin. This cut produced two planar surfaces for each tooth that received experimental treatment: 1) the intaglio surface of the 2 mm flat dentin disc and 2) the flat occlusal surface of the bulk remainder of the tooth. The location and orientation of the cut was chosen to produce a section perpendicular to the dentin tubule direction. The smear layer was removed from all treatment surfaces with 35% phosphoric acid (Fisher Scientific, Waltham, MA, USA) that was applied for 15 sec followed by a 15 sec rinse with DI water. Excess water was removed with a gentle stream of compressed air for 5 sec.

Experimental solutions were applied to three of the dentin discs and the three occlusal surfaces of the bulk remainder of each corresponding tooth. The fourth tooth samples served as controls. Disc/Bulk 1: 42% zinc nitrate was applied to both surfaces using a microbrush with a scrubbing motion for 15 sec. After two min, the sample was immersed in 1×PBS. Disc/Bulk 2: 42% zinc nitrate was applied to both surfaces with a scrubbing motion for 15 sec. After two min, a gentle stream of compressed air was used for 5 sec to remove visible liquid on the surface. Next, 24% dibasic ammonium phosphate solution was applied to both surfaces with a scrubbing motion for 15 sec. After two min, the samples were immersed in 1×PBS solution. Disc/Bulk 3: The same method was used as for disc 2 except that dibasic ammonium phosphate solutions were applied first followed by zinc nitrate. Disc/Bulk 4: No experimental solutions were applied. After removing the smear layer the disc was immediately immersed in a PBS solution.

Dentin samples: Preparation for SEM and EDS analysis. After 24 h the four flat disc samples were removed from the PBS solution and allowed to air dry for three days. The flat discs were adhered to aluminum stubs with carbon paint and then carbon coated using a Desk V carbon evaporator (Denton Vacuum, LLC, Moorestown, NJ USA). The four bulk dentin samples for each tooth were embedded in polymethylmethacrylate and then sectioned in a mesial-distal orientation along the long axis of the tooth and through the center of each tooth. This created dentin cross section samples in which the tubule long axes lay in the surface plane for SEM analyses. Samples were adhered to aluminum stubs with carbon paint and then carbon coated for SEM analysis using a Desk V carbon evaporator (Denton Vacuum, LLC, Moorestown, NJ USA). The intaglio surfaces of the discs were used to investigate the surface of the dentin after treatment. The mesial-distal sectioned tooth was used to investigate the penetration of material into the dentin tubules after treatment.

Milled HAp samples: Mineral formation. Milled HAp coated substrate was treated with several drops of stock zinc nitrate solution delivered from a Pasteur pipette. The solution was gently spread to form a film on the surface of HAp. After a period of 5 min excess solution was wicked away using a Kimwipe. Several drops of stock dibasic ammonium phosphate solution were then added to the sample and allowed to sit for 3 min after which excess solution was wicked away and the sample was washed with DI water and allowed to dry overnight. Dried samples were then characterized by SEM or XRD.

Preparation of zinc phosphates on a planar substrate: Preparation for SEM, EDS, and XRD analysis. For SEM measurements aluminum backed carbon tabs (Ted Pella, 16091-12) was applied to aluminum pin stubs (Ted Pella, 16111-9) platforms. HAp or zinc phosphate in powder form, were applied to the exposed adhesive carbon tab surface. Selected solutions for mineral deposition were applied to the HAp layer by Pasteur pipette. After drying in air samples were sputter coated with gold for SEM and EDS analysis

For XRD measurements flat samples were prepared as surrogates for the native or treated dentin surface. The native surface mimic consisted of HAp powder anchored by epoxy on a Scotch tape protected silicon wafer (001 cut, 1 cm×1 cm). To prepare the HAp substrate liquid 5-min Epoxy (Devcon, clear) was deposited on the tape and milled HAp was tamped on the epoxy with sufficient excess to ensure that the exposed substrate surface was rich in HAp. After drying the assembly was tapped lightly with a spatula to remove unbound HAp and then washed with DI water to further remove loosely bound HAp. The wafer was adhered to a pin stub mount. The mount with the sample was carefully secured to the sample holder in the instrument. Samples of zinc phosphate on HAp were immersed in DI water for one week, after which the samples were washed with DI water followed by acetone and allowed to dry. A second set of samples of zinc phosphate on HAp were immersed in artificial saliva for one week. These samples were then washed with DI water, followed by acetone and then dried before SEM and XRD analysis.

Bulk mineral samples: Effect of solutions on mineral. Two portions of zinc ammonium phosphate powder, each weighing 1 mg were suspended in approximately 1 ml of DI water and 1 ml of artificial saliva respectively for 11 days in test tubes sealed with rubber stoppers. At the end of the soak period the powders were resuspended by sonication, transferred to Eppendorf centrifuge tubes and centrifuged at 8000 rpm for 6 min. The supernatant was discarded. A 1 ml aliquot of DI water was added to each sample followed by vortex mixing and the centrifugation step was repeated. The supernatant was removed and the resuspension/centrifugation step was repeated. Finally, the samples were resuspended in methanol, 1 ml and then twice with ethyl ether, followed in each step with centrifugation. The samples were then dried in air before being packed into 0.5 mm Kapton capillaries and mounted on a magnetic base for powder XRD analysis.

Two samples of zinc phosphate powder, 55 mg in 1 ml DI water, and 60 mg in 1 ml artificial saliva were allowed to stand for 1 week in test tubes sealed with parafilm. Samples were worked up by standard methods and stored in vials after drying. Powder samples were adhered to epoxy on an Si support as described above for HAp.

Dentin samples: SEM images and EDS analysis of the dentin samples were acquired with a Hitachi S-3500N SEM (Hitachi, Tokyo, Japan) in backscattered electron (BSE) mode and a Bruker XFlash 6110 energy dispersive X-ray spectrometer (Bruker, Billerica, MA, USA), respectively. ESPRIT version 2.1 was used to capture BSE and EDS images. All images were taken at high vacuum, 20 kV, with a working distance of 15 mm. For EDS images the following settings were used in the ESPRIT software: Map display filter: Smooth=7, Map color mixing: Enhanced.

Powdered HAp samples: SEM images and EDS analysis of HAp was acquired with a Carl-Zeiss Gemini 300 Field Emission-Scanning Electron Microscope (FE-SEM) and Bruker XFlash 6160, respectively.

Powdered samples were layered onto a zero background Si substrate on a PanAnalytical X'Pert XRD instrument. All scans used a curved crystal monochromator with the following settings: 45 kV, 40 mA, step size 0.013° (2θ) at 99.45 s per step, and a range from 10-45° (PanAnalytical, Malvern Panalytical, Almelo, The Netherlands).

XRD capillary data were collected on a Bruker D8 DISCOVER GADDS microdiffractomer equipped with a VANTEC-2000 area detector in a φ rotation method. The X-ray generated from a sealed Cu tube is monochromated by a graphite crystal and collimated by a 0.5 mm MONOCAP (λ, Cu-Kα=1.54178 Å). The sample detector distance is 150 mm, and the exposure time is 300-1500 sec per run. The sample position was adjusted by a manual control box with the aid of a video camera and alignment laser. Data were integrated by the XRD2EVAL program in the Bruker PILOT software. The raw file was converted by the UXD format by the DIFFRACplus File Exchange, which was later analyzed by the WINPLOTR program.

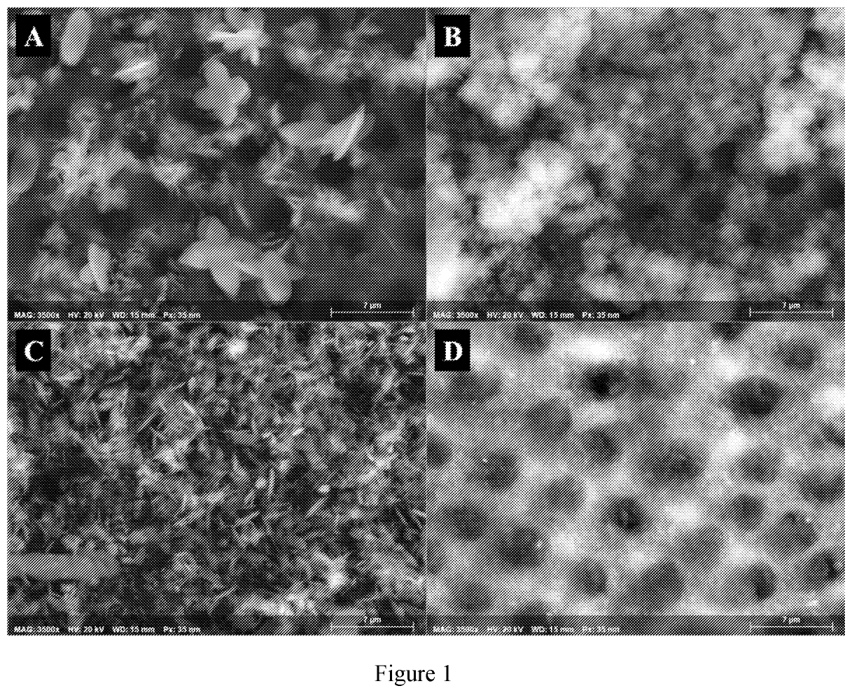

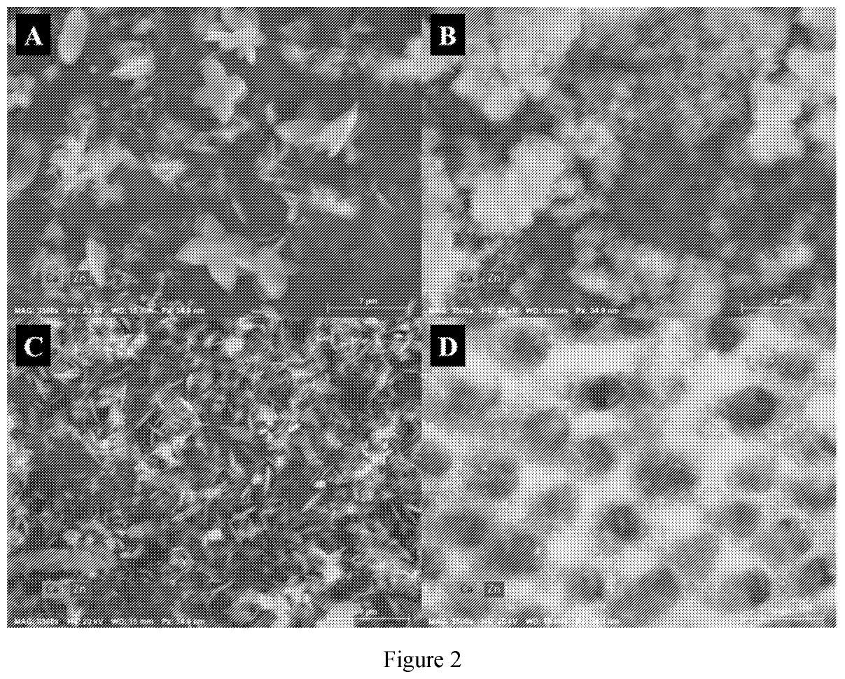

Dentin disc surface samples: SEM/EDS characterization after treatment. The intaglio surfaces of the disc samples were imaged in BSE mode and characterized for element distribution using EDS. Images were acquired at 20 kV, at a working distance of 15 mm, and field widths of 122.88 μm and 35.84 μm. Spectra and elemental quantifications were obtained for all samples. SEM BSE images are seen in FIG. 1 and the corresponding EDS images are seen in FIG. 2. Calcium is labeled with red and zinc is labelled with blue. Phosphorus (not shown) was uniformly detected over the field width of all samples, indicating that phosphorus is present in both the crystals and dentin.

Disc 1: 42% zinc nitrate. Multiple crystal morphologies are observed (FIGS. 1A and 2A) with the largest crystals appearing to be oblong, flat, hexagonally shaped, and up to approximately 6 μm long by 2 μm wide. Other crystals appear as fine spicules approximating 1 μm in length. EDS visually confirmed the presence of zinc in the both crystal species.

Disc 2: 42% zinc nitrate followed by 24% dibasic ammonium phosphate. The dominant species appears (FIGS. 1B and 2B) to be clusters of fine spicules approximating 1 μm in length. Analysis of the spectrum and quantification showed that this surface had the highest relative abundance of zinc by atomic percentage.

Disc 3: 24% dibasic ammonium phosphate followed by 42% zinc nitrate. Crystal morphology was heterogeneous (FIGS. 1C and 2C). There appear to be fine spicules 1-2 μm in length and small flat rectangular species that are generally 1 μm or less in their longest dimension.

Disc 4: Control. No precipitates or zinc was observed on the control surface (FIGS. 1D and 2D).

Dentin bulk samples, cross sectioned to visualize the longitudinal deposition of zinc minerals: SEM/EDS characterization after treatment. The surfaces of the mesial-distal cross-sectioned samples were imaged in BSE mode and characterized with EDS. Images were taken using 20 kV, at a working distance of 15 mm, and at field widths of 204.8 μm and 62.46 μm for each sample. Spectra and elemental quantifications were obtained for all samples. Calcium and zinc are labeled blue and yellow respectively. Phosphorus (not presented) was uniformly detected over the field width of all samples, indicating that phosphorus is present in both the crystals and dentin.

An appreciable amount of intratubular precipitate was detected on tooth 2, and tooth 3. Images for tooth 1 (42% zinc nitrate only) and tooth 4 (control) are not shown. Only a few crystals were detectable on Disc 1 and the furthest penetration was approximately 40 μm from the dentin surface. No precipitate or zinc was detected on the control.

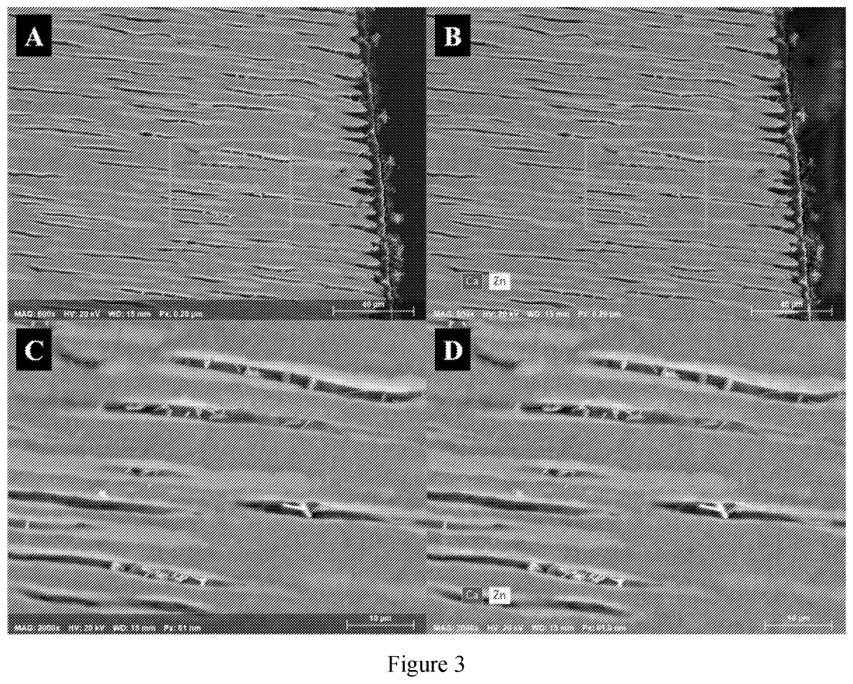

Tooth 2: 42% zine nitrate then 24% dibasic ammonium phosphate. Zinc and phosphorus containing crystals were found in nearly every tubule up to depths of approximately 120 μm (FIG. 3). The morphologies of the crystals seen in the narrow field image appear to be fairly uniform throughout the sample. Most crystals have lengths of 2-3 μm, which closely approximates the width of the dentin tubule in which they are embedded.

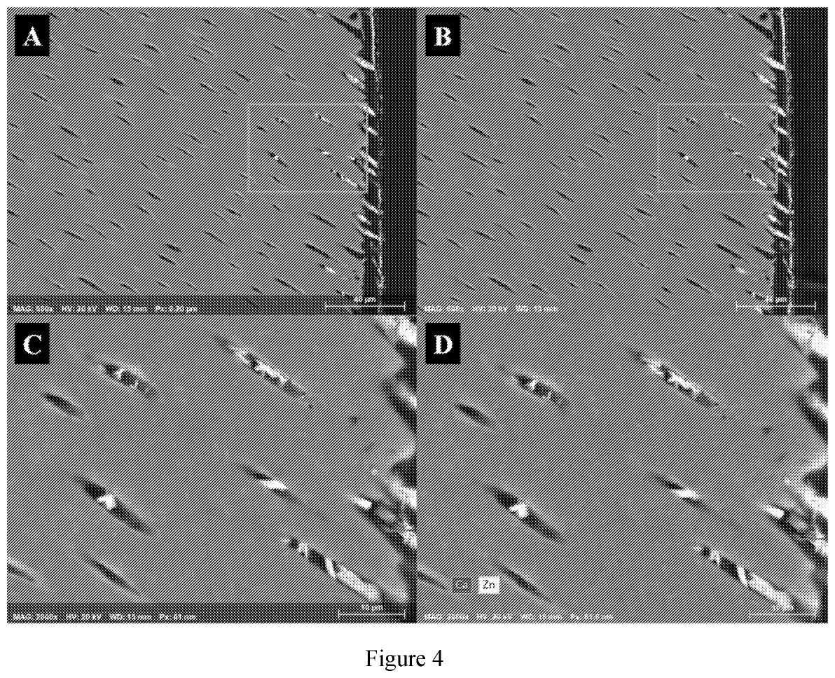

Tooth 3: 24% dibasic ammonium phosphate then 42% zinc nitrate. Zinc and phosphorus containing crystals were found up to 50 μm from the tubule opening (FIG. 4). The majority of crystals were concentrated at the tubule orifice. The crystal species that occlude the tubule openings appear to have a different morphology than the internal crystals.

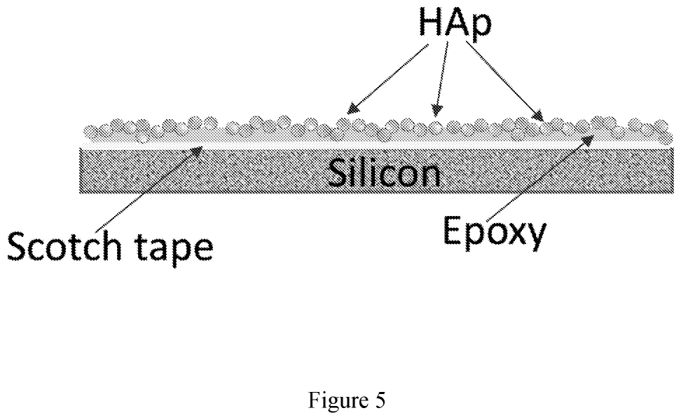

HAp samples: Deposition of zinc phosphate on HAp (zinc phosphate/HAp) XRD and SEM. A silicon plate was protected by applying adhesive tape. A thin layer of epoxy was deposited on the tape surface. Milled HAp was then gently tamped onto the epoxy surface and the epoxy was allowed to set for approximately 30 min (FIG. 5).

Excess HAp was dislodged by tapping the inverted Si support. The presence of unbound excess mineral after epoxy curing ensured that the reactive mineral surface was not covered by adhesive.

Solutions containing zinc and phosphate combine to deposit zinc phosphate minerals. Mineral deposition on HAp was carried out by the sequential addition of 42% zinc nitrate followed by 24% dibasic ammonium phosphate or its reverse. Following the addition of zinc nitrate solution excess liquid was wicked away using a Kimwipe leaving a thin film of solution on HAp. The ammonium phosphate solution was naturally in excess of the zinc nitrate in the liquid film. After 10 min the liquid was again wicked away and the sample was washed with DI water and allowed to dry for at least two hours.

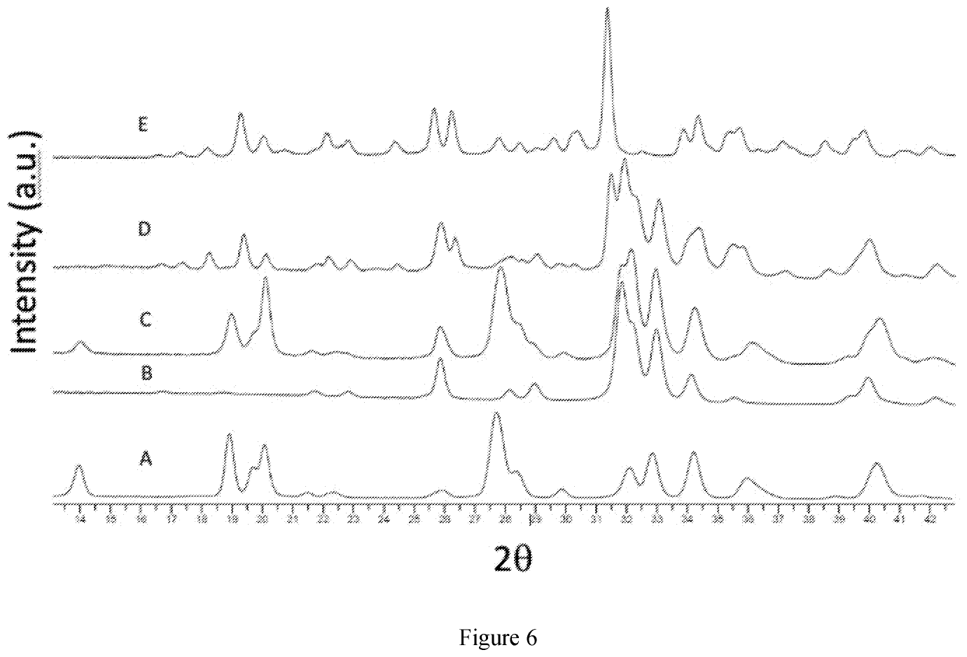

XRD data shows the formation of zinc ammonium phosphate, (NH₄)ZnPO₄, (85444-ICSD) on milled HAp (151414-ICSD) (FIG. 6). Zinc ammonium phosphate peaks appear at 20 values of 14, 19, 20, 28, and 36. Other peaks belong to milled HAp, either alone or overlapping zinc ammonium phosphate. In the reverse order of reagent addition, where 24% dibasic ammonium phosphate was applied to the HAp surface followed by 42% zinc nitrate, the mineral product was hopeite, Zn₃(PO₄)₂·4H₂O, identified its powder pattern (34869-ICSD) (FIG. 6).

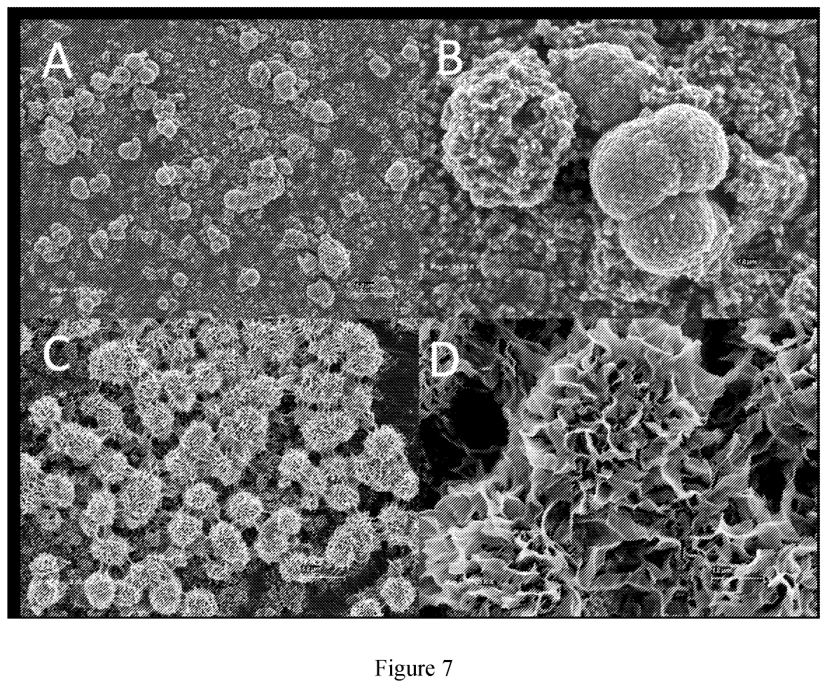

The corresponding SEM images for the two minerals deposited on HAp clearly show the cauliflower-like structure of zinc ammonium phosphate and the flower-like structure of hopeite (FIG. 7).

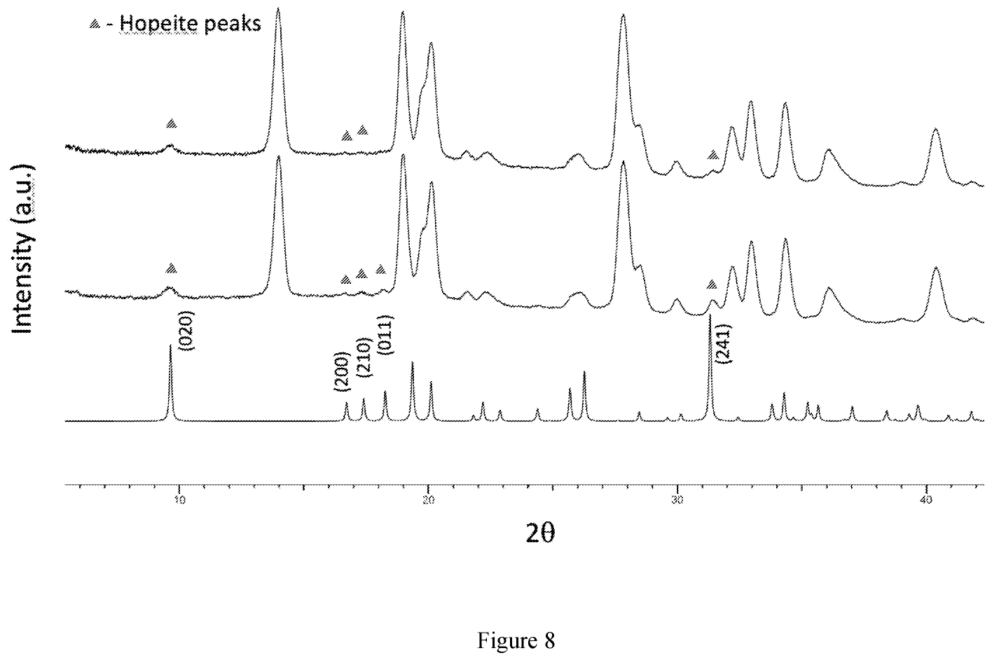

The fate of zinc minerals in aqueous media: Immersion of zinc ammonium phosphate in DI water and artificial saliva. Key to understanding the utility of zinc phosphate in dentin tubules is the nature of its transformation by prolonged exposure to the ion-rich environment of saliva as occurs in patent dentin tubules. Zinc ammonium phosphate powder immersed for 11 days in artificial saliva and water undergoes a transformation to zinc phosphate to different degrees (FIG. 8).

The peaks at 2θ: 17, 17.5, 18 and 31.8 belong to hopeite (34869-ICSD). In this immersion experiment the transformation of zinc ammonium phosphate to the more stable phase, hopeite, is more pronounced in DI water, but negligible in artificial saliva.

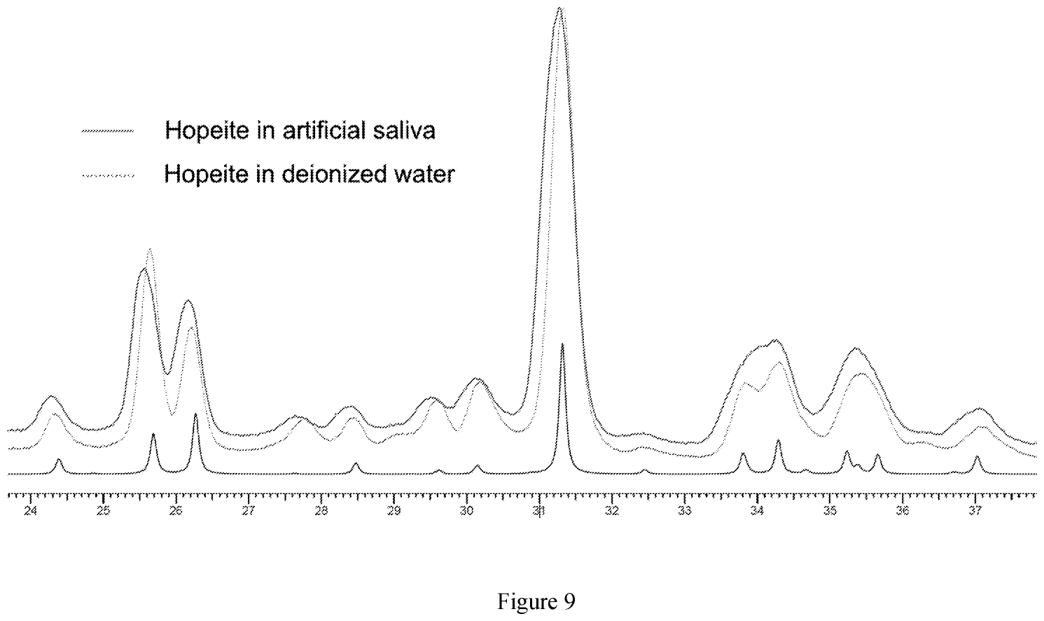

Hopeite does not undergo a morphological change in aqueous media. Its powder diffraction pattern is essentially unchanged except for a peak broadening (FIG. 9). The full width (2θ) at half-maximum for the (241) peak at 31.3 is 0.368 for hopeite in DI water, and 0.487 for hopeite in artificial saliva.

Application of the Scherrer equation,

$$B ( 2 θ ) = \frac{K λ}{L cos θ}$$

(where B is the peak full width at half maximum, K is a constant, 0.89, λ is the radiation wavelength, 0.154 nm, L is the crystallite size and θ is the angle of radiation incidence, 12.0 deg) gives L values of 22.4 nm and 16.8 nm respectively. The apparent crystallite size difference seems to attenuate with time for dry samples, perhaps as a result of changes in hydration.

The foregoing results support the feasibility of using zinc, with known antibacterial properties, for the treatment of dental caries. A dentin block model was employed to study the reaction of aqueous zinc ions with aqueous phosphate ions to deposit zinc phosphate minerals on the dentin surface and at both the openings and the interior of dentin tubules. The result is the rapid occlusion of patent tubules. The formation and the fate of the relevant zinc phosphate minerals in an aqueous environment was examined on a milled HAp surface as a dentin surrogate. That approach facilitated product analysis by XRD and SEM which showed that the order of ion addition determined both the composition of the mineral and the region of its deposition.

The sequential application to the dentin surface of aqueous zinc then phosphate ions results in the uptake of each solution into tubules where they mix to deposit zinc phosphate. SEM and EDS analysis show that the addition sequence zinc then phosphate produces a zinc phosphate mineral in the form of 2-3 micron scale crystals that are fairly uniformly distributed within dentin tubules up to depths of 120 microns. When phosphate solution is applied first, followed by zinc (phosphate-then-zinc) smaller needle-shaped crystals are more prevalent on the dentin surface where they occlude the tubule openings.

The mineral component of peritubular dentin has been shown to be a calcium deficient, carbonated HAp. For simplicity in this study, the deposition of zinc phosphate mineral from combined solutions was evaluated with a milled HAp substrate serving as an analogue of the surface of peritubular dentin.

Parallel experiments, where milled HAp was employed as a surrogate for dentin, provide insight into the conditions for mineral formation and transformation. In this system a layer of HAp was exposed to an aqueous medium while attached to an inert underlying silicon substrate by a layer of epoxy and adhesive tape. The application of zinc salt solution followed by phosphate solution (zinc then phosphate) produced a stable mineral phase, zinc ammonium phosphate, that was attached to HAp. Excess zinc nitrate solution was blotted away leaving a film, hence the following dibasic ammonium phosphate ions were in excess. These conditions favor the formation of the observed product.

(NH₄)₂HPO₄(excess)+Zn(NO₃)₂→(NH₄)ZnPO₄+NH₄⁺+H⁺+2NO₃⁻+n(NH₄)₂HPO₄

When the order of reagent addition is reversed, excess ammonium phosphate solution is wicked away before the addition of zinc nitrate, which nominally leads to a reaction with the stoichiometry:

Zn(NO₃)₂(excess)+2(NH₄)₂HPO₄→Zn₃(PO₄)₂·4H₂O+4NH₄⁺+2H⁺+6NO₃⁻+n Zn(NO₃)₂

Studies on SDF and calcium phosphate show that patent dentin tubules are readily infiltrated with solutions that are applied to the dentin surface. The use of calcium phosphate as an occlusal agent has not been further developed. The result of SDF uptake is the appearance of silver deep within the tubules. The antibacterial efficacy of SDF requires that the silver deposits remain within tubules for extended periods of time for sustained bactericidal function. The deposition of silver from SDF may involve the combined uptake of ions into HAp and the formation of insoluble silver metal nanoparticles under the photoreduction of silver phosphate by ambient light.

The deposition and persistence of zinc requires different conditions from those for silver. Since the direct uptake of zinc ions from solution by HAp is intrinsically low the deposition of zinc is best achieved by the formation of de novo mineral, which is achieved with the formation of zinc phosphates. The deposition of zinc ammonium phosphate within dentin tubules is favored by the specific order of zinc-then-phosphate reagent addition to the dentin surface. This approach produced the greatest depth of tubule penetration (120 microns) by zinc and the highest density of tubule occlusion by crystals.

In aqueous solution zinc ammonium phosphate, (NH₄)ZnPO₄, converts over time to the more stable phase hopeite, Zn₃(PO₄)₂·4H₂O (FIG. 6). This process is diminished in artificial saliva, presumably because phosphate ions produce an equilibrium where the phosphate to zinc ratio is high which favors (NH₄)ZnPO₄ over Zn₃(PO₄)₂. However, in clinical applications it may obtain that hopeite is the ultimate phase that would persist in treated teeth where it would provide a long-term reservoir of zinc. Further, it could undergo ion exchange with calcium ions to produce calcium phosphate phases in dentin tubules that could serve to remineralize carious tissue, a desirable outcome. These results suggest that zinc phosphate can be produced to occlude dentinal tubules, increase dentin hardness, and reduce the permeability of exposed dentin. No discoloration was observed on any of the dentin treated in this experiment. Hopeite is known to have antimicrobial properties. In one application as a titanium implant coating hopeite has been shown to be biocompatible, antibacterial, and a promoter of HAp formation.

These results show that the topical application of zinc and phosphate salt solutions produces mineral deposits on the surface and within the tubules of dentin. Experiments on HAp surfaces allow the identification of colorless zinc phosphate mineral that is deposited as a function of reaction stoichiometry and the nature of mineral transformation in extended contact with aqueous media. The protocol for mineral formation and the nature of the products may prove to be well suited, by their antibacterial activity and their aesthetic acceptability, for the treatment of dental caries or the mitigation of hypersensitivity.

Without intending to be bound by any particular theory, it is considered that the zinc ammonium phosphate precipitate, in particular, will have activity against Streptococcus mutans because it is a source of zinc ions. Additionally, this novel zinc-based dentin treatment opens up a wealth of other areas for investigation including the mechanical effect on resin adhesive bonding, the long-term color stability of dentin after application, and an improved protocol to increase the depth or density of crystal penetration.

### Example 2

A goal of the present disclosure was to deposit (1) zinc phosphate, (2) zinc ammonium phosphate and then (3) zinc fluoride in dentin. Following is a summary of the precursors and products that have been produced:
1) Zn(NO₃)₂·6H₂O+(NH₄)₂HPO₄→Zn(NH₄) PO₄ and Zn₃(PO₄)₂
2) Zn(NO₃)₂·6H₂O+ZnF₂·4H₂O→2[ZnF(H₂O)₅]NO₃,
3) Zn(NO₃)₂·6H₂O+Na₂FPO₃→ZnFPO₃, This product is already known.
4) ZnF₂·4H₂O+NH₃(MeOH)→[Zn(NH₃)₄]F₂,
5) ZnF₂·4H₂O+NH₃(EtOH)→[Zn(NH₃)₄]F₂, expected congruence with reaction 4
6) [Zn(NH₃)₄]F₂+H₂O→ZnO+2(NH₄)F+2NH₃ (aq)
   Zinc tetraammine difluoride has been crystallized and structurally characterized for the first time. The synthesis itself is simple and provides a facile route to the product in a solid crystalline form. The conversion to zinc oxide is very clean, and probably quantitative. This is a key reaction that central to our technology as it potentially delivers simultaneously both zinc oxide, as an antibacterial and fluoride as a remineralization aid.

### Example 3

Also provided is the delivery of ZnO to dentin tubules.

A new method of ZnO deposition from solutions of Zn²⁺ salts in aqueous ammonia is described. The Zn²⁺ salts may be ZnF₂, Zn(NO₃)₂, ZnSO₄, ZnCl₂, ZnBr₂, or ZnI₂ for example. The concentration of ammonia (NH₃) may be 10%, 20%, or 30% by weight. Other percentages may be suitable. The molar ratio NH₃:Zn²⁺ in the solution may be 1:1, 2:1, 3:1, 4:1, 5:1, 6:1, 7:1-10:1.

A new method of ZnO deposition from solutions of Zn²⁺ salts in alcoholic ammonia is described. The alcohol may be methanol, ethanol, N-propanol, isopropanol, butanol, sec-butanol, tert-butanol, propylene glycol, or ethylene glycol. The Zn²⁺ salts may be ZnF₂, Zn(NO₃)₂, ZnSO₄, ZnCl₂, ZnBr₂, or ZnI₂ for example. The concentration of ammonia (NH₃) may be 1N, 2N-N. Other concentrations may be suitable. The molar ratio NH₃:Zn²⁺ in the solution may be 1:1, 2:1, 3:1, 4:1, 5:1, 6:1, 7:1-10:1.

The formation of ZnO occurs upon dilution of Zn²⁺/NH₃(aq) solutions in deionized water. The dilution should be approximately 20× i.e., 50 microliters of solution added to 1 ml of deionized water.

The formation of ZnO occurs upon dilution of Zn²⁺/NH₃(alc) solutions in deionized water. The dilution should be approximately 20× i.e. 50 microliters of alcohol solution added to 1 ml of deionized water.

The formation of ZnO at a carious lesion where Zn²⁺/NH₃(aq) or Zn²⁺/NH₃ (alc) is deposited as an antimicrobial on a carious lesion.

The formation of ZnO at a carious lesion where Zn²⁺/NH₃(aq) or Zn²⁺/NH₃ (alc) is deposited as an as a tubule occluding agent on a carious lesion.

### Example 4

The following data is derived from the crystallographic information for F₂H₁₈N₄O₃Zn.

checkCIF/PLATON Report
Structure factors have been supplied for datablock(s) 22mcw3h

### THIS REPORT IS FOR GUIDANCE ONLY. IF USED AS PART OF A REVIEW PROCEDURE FOR PUBLICATION, IT SHOULD NOT REPLACE THE EXPERTISE OF AN EXPERIENCED CRYSTALLOGRAPHIC REFEREE.

| | | |
|---|---|---|
| No syntax errors found. | CIF dictionary | Interpreting this report |

Datablock: 22mcw3h

| Bond precision: | Zn—N = 0.0018 A Wavelength = 0.71073 | |
|---|---|---|
| Cell: | a = 7.0923(2) b = 7.0923(2) c = 10.9395(5) | |
| | alpha = 90 beta = 90 gamma = 120 | |
| Temperature: | 100 K | |
| | Calculated | Reported |
| Volume | 476.54(3) | 476.54(4) |
| Space group | P 3 1 c | P 3 1 c |
| Hall group | P 3 −2c | P 3 −2c |
| Moiety formula | H12 N4 Zn, 2(F), 3(H2 O) | ? |
| Sum formula | F2 H18 N4 O3 Zn | F2 H18 N4 O3 Zn |
| Mr | 225.57 | 225.55 |
| Dx, g cm−3 | 1.572 | 1.572 |
| Z | 2 | 2 |
| Mu (mm−1) | 2.581 | 2.581 |
| F000 | 236.0 | 236.0 |
| F000′ | 236.69 | |
| h, k, lmax | 9, 9, 14 | 9, 9, 14 |
| Nref | 798[403] | 786 |
| Tmin, Tmax | 0.341, 0.427 | 0.360, 0.480 |
| Tmin′ | 0.265 | |

| | |
|---|---|
| Correction method= | # Reported T Limits: Tmin = 0.360 Tmax = 0.480 |
| AbsCorr= | MULTI-SCAN |
| Data completeness= | 1.95/0.98 |
| Theta(max)= | 28.270 |
| R(reflections)= | 0.0107(778) |
| wR2(reflections)= | 0.0287(786) |
| S= | 1.186 |
| Npar= | 51 |

### Crystal Structure Report for 22mcw3h

A colorless, block-like specimen of F₂H₁₈N₄O₃Zn, approximate dimensions 0.330 mm×0.360 mm×0.500 mm, was used for the X-ray crystallographic analysis. The X-ray intensity data were measured on a Bruker D8 SMART APEXII three-circle diffractometer system equipped with a Incotec microfocus sealed X-ray tube (MoKα, λ=0.71073 Å) and a multilayer optics monochromator.

TABLE 1
Data collection details for 22mcw3h.

| | | | | | | Width/ | | | Wavelength/ | | | |
|---|---|---|---|---|---|---|---|---|---|---|---|---|
| Axis | dx/mm | 2θ/° | ω/° | φ/° | χ/° | ° | Frames | Time/s | Å | Voltage/kV | Current/mA | Temperature/K |
| Phi | 59.295 | 0.00 | 0.00 | 0.00 | 54.76 | 1.00 | 180 | 1.20 | 0.71073 | 50 | 1.0 | 100 |
| Phi | 59.295 | −10.00 | 350.00 | 0.00 | 54.76 | 1.00 | 360 | 1.20 | 0.71073 | 50 | 1.0 | 100 |
| Omega | 59.295 | −16.00 | 344.00 | 45.00 | 54.76 | −0.50 | 360 | 1.20 | 0.71073 | 50 | 1.0 | 100 |
| Omega | 59.295 | −16.00 | 344.00 | 0.00 | 54.76 | −0.50 | 360 | 5.00 | 0.71073 | 50 | 1.0 | 100 |
| Omega | 59.295 | −16.00 | 344.00 | 90.00 | 54.76 | −0.50 | 360 | 5.00 | 0.71073 | 50 | 1.0 | 100 |
| Omega | 59.295 | −16.00 | 344.00 | 180.00 | 54.76 | −0.50 | 360 | 5.00 | 0.71073 | 50 | 1.0 | 100 |
| Omega | 59.295 | −16.00 | 344.00 | 270.00 | 54.76 | −0.50 | 360 | 5.00 | 0.71073 | 50 | 1.0 | 100 |

A total of 2340 frames were collected. The total exposure time was 2.30 hours. The frames were integrated with the Bruker SAINT software package using a narrow-frame algorithm. The integration of the data using a trigonal unit cell yielded a total of 12572 reflections to a maximum θ angle of 28.27° (0.75 Å resolution), of which 786 were independent (average redundancy 15.995, completeness=99.3%, Rᵢₙₜ=4.75%, R_{sig}=1.84%) and 778 (98.98%) were greater than 2σ(F²). The final cell constants of a=7.0923(2) Å, b=7.0923(2) Å, c=10.9395(5) Å, volume=476.54(4) Å³, are based upon the refinement of the XYZ-centroids of 9968 reflections above 20 σ(I) with 6.633°<2θ<56.53°. Data were corrected for absorption effects using the Multi-Scan method (SADABS). The ratio of minimum to maximum apparent transmission was 0.756. The calculated minimum and maximum transmission coefficients (based on crystal size) are 0.3590 and 0.4830.
The structure was solved and refined using the Bruker SHELXTL Software Package, using the space group P 3 1 c, with Z=2 for the formula unit, F₂H₁₈N₄O₃Zn. The final anisotropic full-matrix least-squares refinement on F² with 51 variables converged at R1=1.07%, for the observed data and wR2=2.87% for all data. The goodness-of-fit was 1.186. The largest peak in the final difference electron density synthesis was 0.194 e⁻/Å³ and the largest hole was −0.184 e⁻/Å³ with an RMS deviation of 0.035 e⁻/Å³. On the basis of the final model, the calculated density was 1.572 g/cm³ and F(000), 236 e⁻.

TABLE 2
Sample and crystal data for 22mcw3h.

| | |
|---|---|
| Identification code | 22mcw3h |
| Chemical formula | F₂H₁₈N₄O₃Zn |
| Formula weight | 225.55 g/mol |
| Temperature | 100(2) K |
| Wavelength | 0.71073 Å |
| Crystal size | 0.330 × 0.360 × |
| | 0.500 mm |
| Crystal habit | colorless block |
| Crystal system | trigonal |
| Space group | P 3 1 c |

| | | |
|---|---|---|
| Unit cell dimensions | a = 7.0923(2) Å | α = 90° |
| | b = 7.0923(2) Å | β = 90° |
| | c = 10.9395(5) Å | γ = 120° |

| | |
|---|---|
| Volume | 476.54(4) Å³ |
| Z | 2 |
| Density (calculated) | 1.572 g/cm³ |
| Absorption coefficient | 2.581 mm⁻¹ |
| F(000) | 236 |

TABLE 3
Data collection and structure refinement for 22mcw3h.

| Diffractometer | Bruker D8 SMART APEXII three-circle diffractometer | |
|---|---|---|
| Radiation source | Incotec microfocus sealed X-ray tube | |
| | (MoKα, λ = 0.71073 Å) | |
| Theta range for data collection | 3.32 to 28.27° | |
| Index ranges | −9 <= h <= 9, −9 <= k <= 9, −14 <= l <= 14 | |
| Reflections collected | 12572 | |
| Independent reflections | 786 [R(int) = 0.0475] | |
| Coverage of independent | 99.3% | |
| reflections | | |
| Absorption correction | Multi-Scan | |
| Max. and min. transmission | 0.4830 and 0.3590 | |
| Structure solution technique | direct methods | |
| Structure solution program | SHELXT (Sheldrick 2015) | |
| Refinement method | Full-matrix least-squares on F² | |
| Refinement program | SHELXL-2018/3 (Sheldrick, 2018) | |
| Function minimized | Σ w(Fₒ² − F_{c}²)² | |
| Data/restraints/parameters | 786/7/51 | |
| Goodness-of-fit on F² | 1.186 | |
| Final R indices | 778 data; I > 2σ(I) | R1 = 0.0107, wR2 = 0.0284 |
| | all data | R1 = 0.0111, wR2 = 0.0287 |

| | |
|---|---|
| Weighting scheme | w = 1/[σ²(Fₒ²) + (0.0111P)² + 0.0259P] |
| | where P = (Fₒ² + 2F_{c}²)/3 |
| Absolute structure parameter | 0.058(15) |
| Extinction coefficient | 0.0700(60) |
| Largest diff. peak and hole | 0.194 and −0.184 eÅ⁻³ |
| R.M.S. deviation from mean | 0.035 eÅ⁻³ |

TABLE 4
Atomic coordinates and equivalent isotropic
atomic displacement parameters (Å²) for 22mcw3h.
U(eq) is defined as one third of the trace of the orthogonalized Uᵢⱼ tensor.

| | x/a | y/b | z/c | U(eq) |
|---|---|---|---|---|
| Zn1 | 0.666667 | 0.333333 | 0.56540(3) | 0.00851(12) |
| F1 | 0.000000 | 0.000000 | 0.49514(17) | 0.0133(3) |
| F2 | 0.333333 | 0.666667 | 0.53338(15) | 0.0126(4) |
| O1 | 0.2569(2) | 0.35605(19) | 0.36682(12) | 0.0142(3) |
| N1 | 0.6888(2) | 0.6110(2) | 0.62853(14) | 0.0111(3) |
| N2 | 0.666667 | 0.333333 | 0.3809(2) | 0.0133(5) |

TABLE 5
Bond lengths (Å) for 22mcw3h.

| | | | |
|---|---|---|---|
| Zn1—N1 | 2.0177(15) | Zn1—N1#1 | 2.0177(15) |
| Zn1—N1#2 | 2.0177(15) | Zn1—N2 | 2.019(3) |
| O1—H1D | 0.817(13) | O1—H1E | 0.843(13) |
| N1—H1A | 0.899(13) | N1—H1B | 0.887(13) |
| N1—H1C | 0.893(13) | N2—H2 | 0.898(13) |
| N2—H2#1 | 0.898(13) | N2—H2#2 | 0.898(13) |

Symmetry transformations used to generate equivalent atoms:
#1−y + 1, x − y, z
#2−x + y + 1, −x + 1, z

TABLE 6
Bond angles (°) for 22mcw3h.

| | | | |
|---|---|---|---|
| N1—Zn1—N1#1 | 108.92(5) | N1—Zn1—N1#2 | 108.92(5) |
| N1#1—Zn1—N1#2 | 108.92(5) | N1—Zn1—N2 | 110.02(4) |
| N1#1—Zn1—N2 | 110.02(4) | N1#2—Zn1—N2 | 110.02(4) |
| H1D—O1—H1E | 113.(3) | Za1—N1—H1A | 106.3(15) |
| Zn1—N1—H1B | 113.9(17) | H1A—N1—H1B | 105.(2) |
| Zn1—N1—H1C | 113.0(16) | H1A—N1—H1C | 112.(2) |
| H1B—N1—H1C | 107.(3) | Zn1—N2—H2 | 110.7(18) |
| Zn1—N2—H2#1 | 110.7(18) | H2—N2—H2#1 | 108.3(19) |
| Zn1—N2—H2#2 | 110.7(18) | H2—N2—H2#2 | 108.3(19) |
| H2#1—N2—H2#2 | 108.3(19) | | |

Symmetry transformations used to generate equivalent atoms:
#1−y + 1, x − y, z
#2−x + y + 1, −x + 1, z

TABLE 7
Anisotropic atomic displacement parameters (Å²) for 22mcw3h.
The anisotropic atomic displacement factor exponent
takes the form: −2π²[h² a*² U₁₁ + . . . + 2 h k a* b* U₁₂]

| | U₁₁ | U₂₂ | U₃₃ | U₂₃ | U₁₃ | U₁₂ |
|---|---|---|---|---|---|---|
| Zn1 | 0.00836(13) | 0.00836(13) | 0.00882(15) | 0.000000 | 0.000000 | 0.00418(6) |
| F1 | 0.0121(5) | 0.0121(5) | 0.0158(9) | 0.000000 | 0.000000 | 0.0060(2) |
| F2 | 0.0117(5) | 0.0117(5) | 0.0144(10) | 0.000000 | 0.000000 | 0.0059(2) |
| O1 | 0.0171(6) | 0.0107(5) | 0.0135(6) | −0.0003(5) | −0.0004(5) | 0.0060(5) |
| N1 | 0.0118(6) | 0.0104(6) | 0.0117(7) | 0.0007(5) | −0.0001(5) | 0.0059(5) |
| N2 | 0.0146(7) | 0.0146(7) | 0.0108(11) | 0.000000 | 0.000000 | 0.0073(4) |

TABLE 8
Hydrogen atomic coordinates and isotropic atomic
displacement parameters (Å²) for 22mcw3h.

| | x/a | y/b | z/c | U(eq) |
|---|---|---|---|---|
| H1D | 0.290(4) | 0.458(3) | 0.414(2) | 0.021000 |
| H1E | 0.171(3) | 0.235(3) | 0.399(2) | 0.021000 |
| H1A | 0.732(4) | 0.624(4) | 0.7069(14) | 0.017000 |
| H1B | 0.792(3) | 0.730(3) | 0.593(2) | 0.017000 |
| H1C | 0.565(3) | 0.614(4) | 0.620(2) | 0.017000 |
| H2 | 0.660(3) | 0.212(3) | 0.352(2) | 0.020000 |

Although the present disclosure has been described with respect to one or more particular embodiments and/or examples, it will be understood that other embodiments and/or examples of the present disclosure may be made without departing from the scope of the present disclosure.

1. A method for treating one or more dental caries comprising
applying to the one or more dental caries a composition comprising a zinc salt and
applying to the one or more dental caries a composition comprising a phosphate salt, wherein Zn₃(PO)₄ (and/or its hydrate) and/or (NH₄)ZnPO₄ are formed
or
applying to the one or more dental caries a composition comprising zinc and fluoride.

2. The method of claim 1, wherein the applying is by brushing the composition onto a surface of the tooth having the dental caries.

3. The method of claim 1, wherein the zinc salt is Zn(NO₃)₂, ZnSO₄, Zn(OAc)₂, ZnF₂, ZnCl₂, ZnBr₂, ZnI₂, Zn(NH₃)₄]F₂, or any combination thereof.

4. The method of claim 1, wherein the phosphate salt is (NH₄)₂HPO₄, (NH₄)H₂PO₄, K₃PO₄, K₂HPO₄, KH₂PO₄, (NH₄)₂PO₃F, or any combination thereof.

5. The method of claim 1, wherein the composition comprising the zinc salt is applied prior to the composition comprising the phosphate salt and/or the composition comprising the phosphate salt is applied prior to the composition comprising the zinc salt.

6. (canceled)

7. The method of claim 1, wherein concentration of the zinc salt is 30-50 wt %, relative to the total weight of the composition.

8. (canceled)

9. (canceled)

10. The method of claim 1, wherein the concentration of the phosphate salt is 10-40 wt %, relative to the total weight of the composition.

11. (canceled)

12. (canceled)

13. The method of claim 1, wherein Zn₃(PO)₄ (and/or its hydrate) and/or (NH₄)ZnPO₄ are formed.

14. The method of claim 1, wherein the composition comprising zinc and fluoride comprises [Zn(NH₃)₄]F₂.

15. The method of claim 14, wherein the composition further comprises a liquid medium further comprising an alcohol, a glycol, and/or water.

16. (canceled)

17. (canceled)

18. (canceled)

19. (canceled)

20. (canceled)

21. (canceled)

22. (canceled)

23. (canceled)

24. A composition comprising [Zn(NH₃)₄]F₂.

25. The composition of claim 24, wherein the composition further comprises a liquid medium further comprising an alcohol, a glycol, and/or water.

26. (canceled)

27. (canceled)

28. (canceled)

29. (canceled)

30. (canceled)

31. (canceled)

32. (canceled)

33. (canceled)

34. A method for preparing an inorganic zinc compound comprising:
diluting with water a solution comprising Zn²⁺/NH₃,
wherein the dilution results in the formation of the inorganic zinc compounder, wherein the inorganic zinc compound is ZnO, Zn(OH)₂, and/or Zn(OH)F.

35. The method of claim 34, wherein the solution comprising Zn²⁺/NH₃ is an aqueous ammonia solution.

36. (canceled)

37. The method of claim 34, wherein the concentration of Zn²⁺ is 1 to 5 M.

38. The method of claim 34, wherein the concentration of ammonia is 10 to 30% by weight relative to the total weight of water and ammonia.

39. The method of claim 34, wherein the molar ratio of NH₃:Zn²⁺ in the solution is 1:1-10:1.

40. (canceled)

41. (canceled)

42. (canceled)

43. (canceled)

44. (canceled)

45. (canceled)

46. The method of claim 34, wherein the solution comprising Zn²⁺/NH₃ is applied onto a surface of a tooth or caries of the tooth prior to dilution.

47. (canceled)

48. The method of claim 34, wherein the method is performed in the mouth of an individual.

49. A method for alleviating hypersensitivity in a tooth of an individual, comprising preparing an inorganic zinc compound on or in the tooth, the preparing comprising:
diluting with water a solution comprising Zn²⁺/NH₃,
wherein the dilution results in the formation of the inorganic zinc compounder, wherein the inorganic zinc compound is ZnO, Zn(OH)₂, and/or Zn(OH)F.
